(19) United States
(12) Patent Application Publication
(10) Pub. No.: US 2025/0064751 A1
(43) Pub. Date: Feb. 27, 2025

(54) STABLE FORMULATIONS OF LIPID-ENCAPSULATED RNA

(71) Applicant: Greenlight Biosciences, Inc., Medford, MA (US)

(72) Inventors: Urmi CHHEDA, Medford, MA (US); Lorenzo AULISA, Medford, MA (US); Namita DODWADKAR, Medford, MA (US)

(21) Appl. No.: 18/726,667

(22) PCT Filed: Jan. 9, 2023
(86) PCT No.: PCT/US2023/010407

§ 371 Date: Jul. 3, 2024

Related U.S. Application Data
(60) Provisional application No. 63/297,350, filed on Jan. 7, 2022.

(51) Int. Cl. A61K 9/51; C12N 15/88

(52) U.S. Cl. CPC A61K 9/5123; A61K 9/5115; C12N 15/88

(57) ABSTRACT

The present disclosure provides lipid nanoparticle (LNP)-encapsulated RNA compositions that are stable without sub-zero storage. As described in detail herein, the compositions comprise an LNP-encapsulated RNA and a combination of excipients that stabilize the composition for at least three months or at least six months under refrigerated conditions.

### BACKGROUND

Currently there are authorized RNA vaccines (involving mRNA encapsulated in lipid nanoparticles, or LNPs) with formulations that are stable for up to seven months at −25° C. to −15° C., or for up to 10 weeks at around 4° C. (2° C. to 8° C.). This stability profile complicates global distribution of mRNA formulations, such as the COVID-19 vaccines. Accordingly, LNP-encapsulated RNA formulations that are stable at 4° C. for at least 3 months, and preferably for at least six months, are desperately needed.

### SUMMARY OF THE DISCLOSURE

The present disclosure provides lipid nanoparticle (LNP)-encapsulated RNA compositions that are stable without sub-zero storage or that are stable for extended time periods following removal from sub-zero storage. The compositions comprise an LNP-encapsulated RNA (such as an encapsulated mRNA) and a combination of excipients that stabilize the composition for at least three months or at least six months or more under refrigerated conditions. In other aspects, the disclosure provides methods of treatment with, and uses of, the compositions, including for vaccination against pathogens such as viruses, or delivering other therapeutic RNAs to subjects in need.

The LNP is a lipid delivery vehicle for encapsulated RNA. LNP compositions that find use with the invention are known in the art. For example, the LNPs for encapsulating RNA may comprise a cationic or ionizable lipid, a neutral lipid, a cholesterol or cholesterol moiety, and a PEGylated lipid. The encapsulated RNA in various embodiments may be messenger RNA (mRNA), or may be an RNAi-inducing agent, such as an siRNA or miRNA. Alternatively, the LNP may encapsulate an antisense oligonucleotide (which may comprise RNA nucleotides), a ribozyme, or a gRNA. The RNA in some embodiments may be a self-amplifying RNA. In some embodiments, the RNA is an mRNA encoding a component of an infectious agent, such as a component of a virus, and the mRNA is encapsulated by the LNPs to provide for an mRNA vaccine composition.

In certain aspects and embodiments, the excipients in the composition comprise or consist essentially of an antioxidant, a non-ionic surfactant, a stabilizing agent, and a pH buffer. In certain aspects and embodiments, the excipients in the composition comprise one or more salts that prevent or reduce mRNA hydrolysis, and a pH buffer, and optionally may further comprise one or more non-ionic surfactants, one or more antioxidants and/or stabilizing agents. Compositions in the various aspects and embodiments may also optionally comprise a cryoprotectant.

An exemplary antioxidant is methionine (e.g., L-methionine). In various embodiments, the concentration of methionine (i.e., or salt thereof) in the composition is from about 0.05% to about 1.50% w/v. In some embodiments, the concentration of methionine in the composition is from about 0.075% w/v to about 0.15% w/v. In various embodiments, the antioxidants comprise cysteine. In various embodiments, the concentration of cysteine (i.e., or salt thereof) in the composition is from about 0.05% to about 1.50% w/v. In some embodiments, the concentration of cysteine in the composition is about 0.12% w/v or about 0.13% w/v. In still other embodiments, the antioxidants include both cysteine (e.g., L-cysteine) and methionine (e.g., L-methionine). In various embodiments the antioxidants comprise citric acid. In various embodiments, the concentration of citric acid (i.e., or salt thereof) in the composition is from about 0.05% to about 1.50% w/v. In some embodiments, the concentration of citric acid in the composition is about 0.05%, or about 0.1%, or about 0.15%. In various embodiments, the concentration of ascorbic acid (i.e., or salt thereof) in the composition is from about 0.05% to about 1.50% w/v. In some embodiments, the concentration of ascorbic acid in the composition is about 0.05%, or about 0.1%, or about 0.15%.

In various embodiments, the composition comprises a non-ionic surfactant, such as a polysorbate or a poloxamer. In some embodiments the poloxamer is Poloxamer-188. In some embodiments, the polysorbate is polysorbate-20, polysorbate-40, polysorbate-60, and/or polysorbate-80. In some embodiments, the non-ionic surfactant is polysorbate-20. In various embodiments, the concentration of the non-ionic surfactant is from about 0.001% to about 0.1% w/v or from about 0.005% to about 0.04% w/v (e.g., about 0.01% w/v). Optimal or maximum concentrations of non-ionic surfactant within the ranges described here can be determined for any given RNA concentration. The concentration of non-ionic surfactant in the composition may have a negative effect on encapsulation of RNA if the concentration of the non-ionic surfactant is too high, and such maximum concentration will depend on the concentration of RNA in the formulation.

In various embodiments, the composition comprises a stabilizing agent. In some embodiments, the stabilizing agent is selected from one or more of an amino acid, a polyol, a saccharide, or a hydrolysate, and optionally selected from glycine, sorbitol, and gelatin. In some embodiments, the stabilizing agent is an amino acid, which is optionally glycine (i.e., or salt thereof), which can be present in the composition at a concentration of from about 0.25% to about 15% w/v. In some embodiments, the concentration of such amino acid is about 1.5% w/v. In some embodiments the stabilizing agent is one or more amino acids selected from a charged amino acid, a neutral-hydrophilic amino acid, and a neutral-hydrophobic amino acid. In some embodiments the stabilizing agent is an amino acid selected from one or more of glycine, arginine, lysine proline, histidine, serine, threonine, glutamate, aspartate, glutamine, asparagine, alanine, leucine, valine, and isoleucine. In some embodiments the stabilizing agent is one or more polyols selected from sorbitol and mannitol. In some embodiments the stabilizing agent is one or more saccharide(s) selected from a monosaccharide or disaccharide, and which optionally comprises one or more of pentose and hexose. In some embodiments the saccharide is an aldohexose such as glucose, mannose, or a ketohexose such as fructose. In some embodiments, the saccharide is sucrose or trehalose. In some embodiments the stabilizing agent is one or more hydrolysate of a tissue-derived collagen, such as gelatin. For example, gelatin may be present in the composition at from about 1% to about 20% w/v, such as about 10% w/v. In another example glycine may be present in the composition at from 0.05% to about 15% w/v, or 0.5% to about 10% w/v, or about 1% to about 2%, such as about 1.5%.

In various embodiments the composition comprises an excipient or combination of excipients that prevents or reduces mRNA hydrolysis. In various embodiments, such excipients include salts or combination of salts that prevent or reduce mRNA hydrolysis such as those selected from sodium chloride, potassium chloride, lithium chloride, ammonium chloride, manganese (II) chloride, magnesium chloride, sodium sulfate, potassium sulfate, lithium sulfate, and ammonium sulfate. For example, the concentration of one or more chloride salts, such as potassium chloride and sodium chloride, is from 10 to about 1,000 mM, such as from about 10 mM to about 600 mM, or from about 10 to about 200 mM, or from about 100 mM to about 200 mM. In some embodiments, the concentration of one or more sulfate salts, such as potassium sulfate and sodium sulfate, is up to about 150 mM, such as from about 2.5 mM to about 150 mM, or from about 10 mM to about 75 mM or from about 25 mM to about 75 mM or from about 33 mM to about 75 mM. In certain embodiments, compositions comprising one or a plurality of salts that prevent or reduce mRNA hydrolysis further comprise excipients selected from antioxidants, stabilizing agents, non-ionic surfactants, and cryoprotectants described herein. For example, in some embodiments, the composition further comprises methionine, glycine and/or cysteine. For example, methionine may be present at a concentration of about 0.025% w/v to about 3.0% w/v, or about 0.025% to about 0.5% or about 0.05% to about 0.25% or about 0.1% w/v to about 0.5% w/v (e.g., 0.15% w/v), and/or cysteine may be at a concentration of about 0.05% to about 0.5%, or from about 0.075% to about 0.5%, or from about 0.10% to about 0.5%, or from about 0.10% to about 0.20%, or about 0.10% to about 0.15% w/v; and/or glycine may be present at a concentration of about 1.5% w/v. Such compositions can further comprise a non-ionic surfactant (e.g., polysorbate-20) and pH buffer, and optionally a cryoprotectant such as sucrose.

In various embodiments, the pH is buffered at a pH from about 6.0 to about 8.0. In certain embodiments the PH is buffered at about 6.0, about 7.4, or at about 8.0. In various embodiments, the composition is pH buffered at about pH 7.4. In various embodiments, the pH buffer is a phosphate buffer or a Tris-EDTA (TE) buffer. In other embodiments the pH buffer is a histidine buffer.

In some embodiments, the composition further comprises an excipient that reduces degradation of the RNA by free-radical oxidation. In some embodiments, the excipient that reduces degradation of the RNA by free-radical oxidation is one or more of ethanol and histidine. In some embodiments, ethanol is included as an excipient at 200 mM or less to avoid effects on LNP size. In some embodiments, the excipient(s) that reduce degradation of the RNA comprise or consist of histidine, which is optionally present in the composition at a concentration of from about 0.01% w/v to about 1% w/v.

In exemplary embodiments, the composition comprises, consists essentially of, or consists of the following excipients in buffered solution: methionine from about 0.05% to about 1.50% w/v; polysorbate-20 from about 0.001% to about 0.1% w/v; and glycine from about 0.25% to about 15% w/v. In exemplary embodiments, the composition comprises, consists essentially of, or consists of the following excipients in buffered solution: methionine from about 0.10% to about 0.25% w/v; polysorbate-20 from about 0.005% to about 0.05% w/v; and glycine from about 0.5% to about 2.5% w/v. In exemplary embodiments, the composition comprises, consists essentially of, or consists of the following excipients in buffered solution: methionine at about 0.15% w/v, polysorbate-20 at about 0.01% w/v, and glycine at about 1.5% w/v.

In some embodiments, the composition comprises, consists essentially of, or consists of the following excipients, in buffered solution, potassium sulfate, sodium chloride, potassium chloride, L-cysteine, L-methionine, and polysorbate-20, each optionally at concentrations described herein (e.g., in about concentrations listed in Tables 10 and Table 11). In some embodiments, the composition further comprises a cryoprotectant, which is optionally sucrose. In some embodiments, the buffer is a Tris-EDTA buffer (TE buffer), optionally 1× TE buffer through 5× TE buffer.

In some embodiments, the composition comprises, consists essentially of, or consists of the following excipients, in buffered solution, potassium sulfate, sodium sulfate, sodium chloride, potassium chloride, L-cysteine, and polysorbate-20, each optionally at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, potassium sulfate, sodium chloride, potassium chloride, L-methionine, and polysorbate-20, each optionally at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, sodium chloride, L-cysteine, and polysorbate-20, each optionally at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, and polysorbate-20, each optionally at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, sodium chloride, potassium chloride, and polysorbate-20, each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium chloride, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, cysteine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium chloride, potassium chloride, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, potassium sulfate, sodium chloride, potassium chloride, cysteine, methionine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride potassium chloride, cysteine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, cysteine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, sodium chloride, potassium chloride, cysteine, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, cysteine, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, cysteine, methionine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, potassium chloride, cysteine, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, cysteine, methionine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, cysteine, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, potassium sulfate, sodium chloride, potassium chloride, methionine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, potassium chloride, cysteine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, cysteine, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, potassium sulfate, sodium chloride, potassium chloride, cysteine, methionine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, cysteine, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, cysteine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium chloride, potassium chloride, cysteine, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, potassium chloride, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, potassium sulfate, sodium chloride, cysteine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, potassium sulfate, sodium chloride, cysteine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium chloride, potassium chloride, cysteine, methionine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, sodium sulfate, sodium chloride, potassium chloride, methionine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises, consists essentially of, or consists of the following excipients, in buffered solution, potassium sulfate, sodium chloride, potassium chloride, cysteine, glycine, and polysorbate-20 each at concentrations described herein (e.g., in about concentrations listed in Tables 10 and Table 11). In some embodiments, the composition further comprises a cryoprotectant, which is optionally sucrose. In some embodiments, the buffer is TE buffer, optionally 1× TE buffer through 5× TE buffer.

In some embodiments, the composition comprises or consists of excipients that are listed in Tables 10 and 11. For example, in some embodiments, each excipient present in the composition is shown in Table 10 or Table 11. The excipient are present at about a concentration shown for that excipient in any sample shown in Table 10 or Table 11, or alternatively, any concentration or concentration range for that excipient as described herein.

In some aspects, the disclosure provides a method for preventing or reducing the probability of a viral infection in a patient or a population (including an animal subject or population), such as but not limited to SARS-COV-2 infection or influenza infection. In these embodiments, the method comprises administering an mRNA vaccine of the present disclosure encoding one or more viral proteins. In accordance with the various aspects, the present disclosure provides for simplified global distribution over currently available mRNA vaccines, since sub-zero conditions are not required for storage and distribution and/or because stability of the vaccine is improved. The disclosure further provides for treatment of other conditions with therapeutic RNA compositions according to the disclosure.

### DESCRIPTION OF THE FIGURES

FIG. 1 shows an initial evaluation of excipients based on % mRNA encapsulation at 4° C. and 25° C. for two days. Description of samples is shown in Table 2. Samples 2, 4, 5, 8, 10 15, 18, and 19 showed significantly less stability than the control (Sample 1).

FIG. 2 shows the size of LNPs (nm) after 2 days at 4° C. or 25° C. Samples 2, 8, and 10 showed a significant increase in size from the control (Sample 1). Description of samples is shown in Table 2.

FIG. 3A is a chromatogram showing intact and degraded mRNA after three months and six months at 4° C.

FIG. 3B shows normalized percent mRNA integrity after three months and six months at 4° C. Description of samples is shown in Table 3.

FIG. 4A shows LNP size (nm) and PDI after three months or six months at 4° C. Description of samples is shown in Table 3.

FIG. 4B shows % mRNA encapsulation after three months or six months at 4° C. Description of samples is shown in Table 3.

FIG. 5A shows normalized % mRNA integrity (against Day 0) for Samples T43-9 and T43-12. Samples were tested after 3, 4.5 and 6 months at 25° C.

FIG. 5B shows luciferase expression after 3, 4.5 and 6 months at 25° C. for Samples T43-9 and T43-12.

FIG. 6A shows % mRNA integrity (normalized to Day 0) over time at 37° C. after LNP-encapsulated mRNAs were diluted in various buffers. Samples are shown in Table 5.

FIG. 6B shows % mRNA integrity of naked mRNA overtime at 37° C. in various buffers. Samples are shown in Table 6.

FIG. 7A shows normalized % mRNA integrity of stable product at 4° C., at time 0 and after 2 months, 3 months, 4.5 months, and 6 months. Formulations were Sample 66 and Sample 67.

FIG. 7B shows normalized % mRNA integrity of stable product at 25° C. at time 0 and after 1 month, 2 months, and 3 months. Formulations were Sample 66 and Sample 67.

FIG. 7C shows normalized % mRNA integrity of stable product at 37° C. at time 0 and after 3 weeks, and 1 month. Formulations were Sample 66 and Sample 67.

FIG. 7D shows LNP size (nm) and PDI after 3, 4.5, or 6 months at 4° C. Formulations were Sample 66 and Sample 67.

FIG. 7E shows % mRNA encapsulation after 3, 4.5, or 6 months at 4° C. Formulations were Sample 66 and Sample 67.

FIG. 8 shows normalized % mRNA integrity for T73-4 and T43-10 at 37° C. at time 0 and after 2 weeks, 3 weeks, and 4 weeks.

FIG. 9 shows normalized % mRNA integrity for samples T117-c-3 and T117-c-5 at 25° C. at time 0 and after 1 month, 2 months, and 3 months.

FIG. 10 shows the effect of various concentrations of methionine as in Table 8 on oxidation of ionizable lipid (ALC-315) using principle of Fenton reaction.

FIG. 11A shows fold change for mRNA integrity in compositions containing hydrophobic non-polar amino acids vs. Tris-EDTA buffer (TE) control after 1 day at 60° C.

FIG. 11B shows fold change for mRNA integrity in compositions containing hydrophilic polar uncharged amino acids vs. TE buffer control after 1 day at 60° C.

FIG. 11C shows fold change for mRNA integrity in compositions containing polar basic amino acids and their derivatives vs. TE buffer control after 1 day at 60° C.

FIG. 11D shows fold change for mRNA integrity in compositions containing polar negatively charged amino acids vs. TE buffer control after 1 day at 60° C.

FIG. 11E and 11F show fold change for mRNA integrity in compositions containing salts and other excipients vs. TE buffer control after 1 day at 60° C.

FIG. 11G shows fold change for mRNA integrity in compositions containing sugars or non-ionic surfactants vs. TE buffer control after 1 day at 60° C.

FIG. 12 shows normalized % mRNA integrity after 2 weeks at 40° C. for various compositions containing excipients selected from NaCl, KCl, Na₂SO₄, K₂SO₄, Cysteine, Methionine, Glycine, Sucrose, and/or Polysorbate-20 in TE and/or PBS buffer shown in Table 10.

FIG. 13 shows normalized % mRNA integrity after 2 weeks at 40° C. for various compositions containing excipients selected from NaCl, KCl, Na₂SO₄, K₂SO₄, Cysteine, Methionine, Glycine, Sucrose, and/or Polysorbate-20 in TE and/or PBS buffer shown in Table 11.

FIG. 14 shows normalized % mRNA integrity through 4 weeks at 37° C. for various concentrations of sodium chloride in TE Buffer as shown in Table 12.

FIG. 15 shows normalized % mRNA integrity through 4 weeks at 37° C. for samples employing various concentrations of TE buffer (Table 13).

### DETAILED DESCRIPTION

The present disclosure provides lipid nanoparticle (LNP)-encapsulated RNA compositions that are stable without sub-zero storage or that are stable for extended time periods following removal from sub-zero storage. As described in detail herein, the compositions comprise an LNP-encapsulated RNA and a combination of excipients that stabilize the composition for at least three months or at least six months or more under refrigerated conditions.

As used herein, the terms “stable”, “stability”, and “stabilize” mean that at least 50% (as measured by HPLC) of the RNA remains intact (not degraded), as compared to the same composition at a time zero.

For example, in various embodiments, the composition is stable for at least three months at 2° C., or is stable for at least six months at 2° C., or is stable for at least twelve months at 2° C. In some embodiments, the composition is stable for at least three months at 4° C., or is stable for at least six months at 4° C., or is stable for at least twelve months at 4° C. In some embodiments, the composition is stable for at least three months at 8° C., or is stable for at least six months at 8° C., or is stable for at least twelve months at 8° C. In various embodiments, the composition is stable for at least six months with refrigeration in the range of 2° C.-8° C. The compositions can therefore be stored and/or distributed at temperatures in the range of 2-8° C., providing substantial advantages over currently authorized mRNA vaccines.

In various embodiments, at least 60% of the RNA remains intact after 6 months at 2-8° C., as compared to the same composition at time zero. In some embodiments, at least 70% of the RNA remains intact after 6 months at 2-8° C., as compared to the same composition at time zero. In some embodiments, at least 80% of the RNA remains intact after 6 months at 2-8° C., as compared to the same composition at time zero.

Degradation or instability of compositions can be further determined by an increase or decrease in average size of the particles in the formulation (e.g., an average size that is at least about 20% or at least about 10% larger or smaller than controls). In various embodiments, degradation or instability of compositions can be determined by dynamic light scattering (DLS). In some embodiments, the average diameter of the LNPs is from about 40 nm to about 110 nm. In some embodiments, the LNPs have an average diameter of from about 50 nm to about 100 nm, or about 50 nm to about 90 nm (e.g., from about 55 to about 80 nm). In various embodiments, the LNPs have an average diameter of about 60 nm, about 65 nm, about 70 nm, about 75 nm, about 80 nm, about 85 nm, about 90 nm, about 95 nm, or about 100 nm.

In various embodiments, the population of LNP encapsulating the RNA is relatively homogenous, as determined by a polydispersity index (PDI), which indicates the particle size distribution of the lipid nanoparticles. A small (e.g., less than 0.3) PDI generally indicates a narrow particle size distribution. A LNP may have a PDI from about 0 to about 0.25. In some embodiments, the PDI is from about 0.10 to about 0.20.

In various embodiments, the LNPs in the compositions have relatively low charges, positive or negative, as more highly charged species may interact undesirably with cells or tissues in the body upon administration. In some embodiments, the zeta potential of a the LNPs in the composition may be from about −20 mV to about +20 mV, or from about −10 mV to about +10 mV.

RNA degradation can be determined by the presence of smaller RNA species and disappearance of the desired RNA size, as determined for example by high performance liquid chromatography (HPLC). See, e.g., Packer M. et al., A novel mechanism for the loss of mRNA activity in lipid nanoparticle delivery systems. Nature Communications vol. 12, Article 6777 (2021).

In certain aspects and embodiments, the excipients in the composition comprise or consist essentially of an antioxidant, a non-ionic surfactant, a stabilizing agent, and a pH buffer. In certain aspects and embodiments, the excipients in the composition comprise or consist essentially of a non-ionic surfactant, one or more salts that prevent or reduce mRNA hydrolysis, and a pH buffer, and optionally may further comprise one or more antioxidants and/or stabilizing agents. Compositions in the various embodiments may also further optionally comprise a cryoprotectant. In this context, the term “consist essentially of” means that additional excipients (including those described herein) can be included in the composition that do not affect stability of the LNP-encapsulated mRNA compared to the reference composition, which can be determined using analytical assays described herein.

Exemplary antioxidants include methionine (e.g., L-methionine), ascorbic acid (Vitamin C)/ascorbate, citric acid/citrate, monothioglycerol, phosphoric acid, potassium metabisulfite, alpha-tocopherol, sodium sulfite, cysteine (e.g., L-cysteine), sodium metabisulfite, L-cysteine-HCl, vitamin E TPGS (i.e., D-α-tocopheryl polyethylene glycol succinate), 2-Hydroxypropyl-β-cyclodextrin (i.e., HP-β-CD), Butylated hydroxyanisol (BHA), Butylated hydroxytoluene (BHT), vitamin A, or other antioxidant approved for injectable use in humans. Further exemplary antioxidants, such as polyphenols or vitamin P, not yet approved for injectable use, may also be used. In the various embodiments the concentration of the antioxidant is from about 0.05% to about 1.50% w/v, or from about 0.05% to about 0.5% w/v, or from about 0.05% to about 0.25% w/v, or from about 0.05% to about 0.15% w/v.

In various embodiments, the antioxidant is methionine (e.g., L-methionine). In various embodiments, the concentration of methionine (i.e., or salt thereof) in the composition is from about 0.05% to about 1.50% w/v, or from about 0.05% to about 0.5% w/v, or from about 0.05% to about 0.15% w/v. In some embodiments, the concentration of methionine in the composition is from about 0.075% w/v to about 0.15% w/v.

In some embodiments, the composition comprises ascorbic acid (i.e., or salt thereof), which can be present in the composition from about 0.05% to about 1.5% w/v, or from about 0.05% to about 0.5% w/v, or from about 0.10% to about 0.25% w/v, or about 0.1% in some embodiments.

In various embodiments, the antioxidant is cysteine. In various embodiments, the concentration of cysteine (i.e., or salt thereof) in the composition is from about 0.05% to about 1.50% w/v, or from about 0.05% to about 0.5%, or from about 0.10% to about 0.45% w/v. In some embodiments, the concentration of cysteine in the composition is about 0.12% w/v or about 0.13% w/v.

In still other embodiments, the antioxidants include both cysteine (e.g., L-cysteine) and methionine (e.g., L-methionine), optionally at concentrations described above. Alternatively, the total amount of L-cysteine and L-methionine is from about 0.05% to about 1.50% w/v, or from about 0.05% to about 0.5%, or from about 0.05% to about 0.25% w/v.

In still other embodiments, the composition does not comprise an antioxidant.

In various embodiments, the composition comprises a non-ionic surfactant, such as a polysorbate or a poloxamer. In some embodiments, the polysorbate is polysorbate-20, polysorbate-40, polysorbate-60, and/or polysorbate-80. In some embodiments, the non-ionic surfactant is polysorbate-20. In various embodiments, the concentration of the non-ionic surfactant such as polysorbate-20 is from about 0.001% to about 0.1% w/v, or from about 0.005% to about 0.05% w/v, or about 0.01% w/v. In some embodiments the poloxamer is Poloxamer 188, Poloxamer 124, Poloxamer 182, Poloxamer 331, Poloxamer 335, Poloxamer 407, or other Poloxamer. In various embodiments, the concentration of poloxamer is from about 0.001% to about 0.1% w/v, or from about 0.005% to about 0.05% w/v, or about 0.01% w/v.

In various embodiments, the composition comprises a stabilizing agent. In some embodiments, the stabilizing agent is selected from one or more of glycine, sorbitol, and gelatin. In some embodiments, the stabilizing agent is glycine (i.e., or salt thereof), which can be present in the composition at a concentration of from about 0.25% to about 15% w/v, of from about 0.5% to about 15% w/v, or from about 0.25% to about 10% w/v, or from about 0.25% to about 5% w/v, or from about 0.5% to about 2.5% w/v. In some embodiments, the concentration of glycine is about 1.5% w/v. In these or other embodiments the stabilizing agent is sorbitol, which is optionally present in the composition at from about 1% to about 20%, such as about 10% w/v. In these or other embodiments the stabilizing agent(s) comprise gelatin, which is optionally present in the composition at from about 1% to about 20% w/v, or from about 5% to about 15% w/v, such as about 10% w/v. In still other embodiments, the composition does not comprise such a stabilizing agent.

In various aspects and embodiments, the composition comprises an excipient or combination of excipients that prevents or reduces mRNA hydrolysis. In various embodiments, such excipients include salts or combination of salts that prevent or reduce mRNA hydrolysis such as those selected from sodium chloride, potassium chloride, lithium chloride, ammonium chloride, manganese (II) chloride, magnesium chloride, sodium sulfate, potassium sulfate, lithium sulfate, and ammonium sulfate. In some embodiments, the one or more salts comprise or consist essentially of those selected from potassium sulfate, sodium sulfate, sodium chloride, and potassium chloride. In some embodiments, the one or more salts comprise at least one chloride salt and at least one sulfate salt. In some embodiments, the one or more salts comprise at least one potassium salt and at least one sodium salt. In some embodiments the one or more salts comprise or consist essentially of potassium sulfate, potassium chloride, and sodium chloride. In some embodiments the one or more salts comprise or consist essentially of sodium sulfate and sodium chloride. In some embodiments, the one or more salts comprise or consist essentially of sodium sulfate, potassium chloride, and sodium chloride. In some embodiments the one or more salts comprise or consist essentially of a combination of salts set forth in the embodiments described in Table 10 or Table 11 (including at concentrations listed in Tables 10 or 11, ±10%). In various embodiments, the concentration of one or more chloride salts, such as potassium chloride and sodium chloride, is from about 10 mM to about 1,000 mM (each), or optionally up to 200 mM, such as from about 100 mM to about 200 mM, or from about 136 mM to about 200 mM. In embodiments, the concentration of one or more sulfate salts, such as potassium sulfate and sodium sulfate, is up to 150 mM (each), such as from about 2.5 mM to about 150 mM, or from about 25 mM to about 75 mM or from about 33 mM to about 75 mM. In various embodiments the concentration of the one or more chloride salts is within the range of such concentrations as set forth in the embodiments described in Table 10 or Table 11. The concentrations of such salts may be a concentration listed in Table 10 or Table 11 (±10%).

In exemplary embodiments, the composition comprises an excipient or combination of excipients that prevents or reduces mRNA hydrolysis, where, for example, the composition has a concentration of potassium sulfate of about 43 mM, a concentration of sodium sulfate of about 34 mM, a concentration of sodium chloride of about 136 mM, and a concentration of potassium chloride of about 136 mM. Alternatively, the composition has a concentration of potassium sulfate of about 75 mM, a concentration of sodium sulfate of about 75 mM, a concentration of sodium chloride of about 200 mM, and a concentration of potassium chloride of about 200 mM. Alternatively, the composition has a concentration of potassium sulfate of about 33 mM, a concentration of potassium chloride of about 199 mM, and a concentration of sodium chloride of about 190 mM. Alternatively still, the composition has a concentration of potassium sulfate of about 47 mM, a concentration of potassium chloride of about 180 mM, and a concentration of sodium chloride of about 141 mM. Alternatively, the composition has a concentration of sodium sulfate of about 48 mM and a concentration of sodium chloride of about 192 mM. In some embodiments, the composition has a concentration of sodium sulfate of about 75 mM, a concentration of potassium chloride of about 125 mM, and a concentration of sodium chloride of about 200 mM.

In certain embodiments, compositions comprising one or a plurality of salts that prevent or reduce mRNA hydrolysis further comprise excipients selected from antioxidants, stabilizing agents, non-ionic surfactants, and cryoprotectants as already described. In some embodiments, the composition does not comprise an antioxidant (e.g., as already described) and/or stabilizing agent (e.g., as already described). In some embodiments, the composition comprises methionine and/or cysteine, such as in concentrations already described. Such salts can be formulated with a non-ionic surfactant (e.g., polysorbate-20, polysorbate-40, polysorbate-60, and polysorbate-80) and pH buffer, and optionally a cryoprotectant (e.g., sucrose). In some embodiments, the non-ionic surfactant is polysorbate-20, which is optionally present at 0.01% w/v. Such compositions are further described in Tables 10 and 11.

In various embodiments, the pH is buffered at a pH from about 6.0 to about 8.0. In certain embodiments the pH is buffered at about 6.0, about 7.4, or at about 8.0. In various embodiments, the composition is pH buffered at about pH 7.4. In various embodiments, the pH buffer is a phosphate buffer. In still other embodiments, the pH buffer is a Tris-EDTA (TE) buffer. In such embodiments the TE buffer is selected from a 1× TE through 10× TE or optionally from 1× TE through 5× TE, or optionally from 1× TE to 3× TE. In some such embodiments the TE buffer is 1× TE buffer. In some embodiments the pH buffer is a histidine buffer. In some embodiments, the histidine buffer is L-Histidine. In some embodiments the buffer is a TE buffer, including Tris HCl and disodium EDTA. In some embodiments the buffer is tris acetate, including tris base and sodium acetate. In some embodiments, the buffer is sodium citrate buffer, which includes sodium citrate dihydrate and citric acid. In some embodiments, the buffer is PBS, which may include potassium chloride, monobasic potassium phosphate, sodium chloride, and dibasic sodium phosphate dihydrate. In some embodiments the PBS is DPBS which may include sodium chloride, potassium chloride, Na₂HPO₄, KH₂PO₄, CaCl₂, and MgCl₂-6H₂O. A 1× TE buffer contains about 10 mM Tris-HCl and about 1 mM disodium EDTA.

In some embodiments, the composition comprises a metal ion chelator. For example, the chelator may be selected from ethylenediaminetetraacetic acid (EDTA), diethylenetriamine pentaacetic acid (DTPA), ethylene glycol-bis(β-aminoethyl ether)-N,N,N′,N′-tetraacetic acid (EGTA), iminodisuccinic acid, polyaspartic acid, ethylenediamine-N,N′-disuccinic acid (EDDS), methylglycine diacetic acid (MGDA), L-glutamic acid N,N-diacetic acid (GLDA), or a salt thereof. In some embodiments, the metal ion chelator is EDTA or salt thereof, which is optionally disodium EDTA. In some embodiments, the concentration of EDTA or disodium EDTA is from about 0.01 mM to about 1 mM, or from about 0.05 mM to about 0.5 mM, or about 0.1 mM.

In various embodiments, the composition further comprises an excipient that reduces exposure of the RNA to water. In some embodiments, the excipient that reduces exposure of the mRNA to water is a saccharide, such as sucrose, dextrose, and/or trehalose. In some embodiments, such excipients act as cryoprotectant. In some embodiments, the composition comprises sucrose, which may be present at from about 1% w/v to about 20% w/v, such as about 15% w/v.

In some embodiments, the composition further comprises an excipient that reduces degradation of the RNA by free-radical oxidation. In some embodiments, the excipient that reduces degradation of the RNA by free-radical oxidation is one or more of ethanol and histidine. In some embodiments, ethanol is included as an excipient at 200 mM or less, or about 150 mM or less, or about 100 mM of less, or about 50 mM or less, to avoid effects on LNP size. In some embodiments, the excipient(s) that reduce degradation of the RNA comprise or consist of histidine. Histidine may be present in the composition at a concentration of from about 0.01% w/v to about 1% w/v, or from about 0.05% w/v to about 0.5% w/v, or about 0.1% w/v.

In some embodiments, the composition does not require, and thus need not comprise, a cryoprotectant. For example, where the product will not be frozen for storage prior to use, then a cryoprotectant need not be included in the composition. The improved stability of the product may in some instances obviate any need to freeze the product, thereby eliminating the usefulness of or need for a cryoprotectant in the composition. A “cryoprotectant” is an excipient that is intended to reduce damage to the composition from freezing. Cryoprotectants include polyols (e.g., propylene glycol, glycerol, ethylene glycol, or diethylene glycol), polymers such as polyethylene glycol (PEG), organic solvents (e.g., dimethyl sulfoxide (DMSO)), and sugars such as sucrose, sorbitol, trehalose, dextrose, erythritol, and xylitol. In other embodiments, the composition may include a cryoprotectant, such as, for example, where the product may be initially frozen but post-thaw requires the improved stability at higher temperatures provided by the present invention. Exemplary cryoprotectants that are conventionally employed (and which are excluded from the composition in some embodiments where the product will not be frozen or included where the product will be frozen for some amount of time) include glycerol, sorbitol, mannitol, sucrose, ethanol, and propylene glycol. In some embodiments the composition comprises a cryoprotectant selected from such conventionally employed cryoprotectants and in embodiments may comprise sucrose.

In exemplary embodiments, the composition comprises, consists essentially of, or consists of the following excipients in buffered solution: L-methionine from about 0.05% to about 1.50% w/v; polysorbate-20 from about 0.001% to about 0.1% w/v; and glycine from about 0.25% to about 15% w/v. In exemplary embodiments, the composition comprises, consists essentially of, or consists of the following excipients in buffered solution: L-methionine from about 0.10 to about 0.25% w/v; polysorbate-20 from about 0.005% to about 0.05% w/v; and glycine from about 0.5% to about 2.5% w/v. In exemplary embodiments, the composition comprises, consists essentially of, or consists of the following excipients in buffered solution: L-methionine at about 0.15% w/v, polysorbate-20 at about 0.01% w/v, and glycine at about 1.5% w/v. In this context, the term “consists essentially of” means that any further excipients included in the composition do not reduce the integrity of the mRNA upon storage at 4° C. for three months, as compared to the reference composition.

In some embodiments, the composition comprises, consists essentially of, or consists of the following excipients, in buffered solution, potassium sulfate, sodium chloride, potassium chloride, L-cysteine, L-methionine, and polysorbate-20, each optionally at concentrations already described (or as described in Table 10 or Table 11). In certain such embodiments the potassium sulfate is in a concentration of about 2.5 mM to about 150 mM, or from about 10 mM to about 75 mM, or from about 25 mM to about 75 mM, or about 33 mM to about 75 mM, or about 33 mM or about 75 mM. In certain of such embodiments the chloride salts are each in a concentration of about 10 mM to about 1,000 mM, or is from about 50 mM to about 600 mM, or is up to about 200 mM, or from about 100 mM to about 200 mM, or from about 136 mM to about 200 mM. In certain of such embodiments the sodium chloride is in a concentration of about 190 mM and the potassium chloride is in a concentration of about 199 mM. In some embodiments, the composition further comprises a cryoprotectant, which is optionally sucrose. In certain of such embodiments the L-cysteine is at a concentration of about 0.1% w/v to about 1.5% w/v or is about 0.12% w/v. In certain embodiments the L-methionine is at a concentration of about 0.15% w/v. In some embodiments, the buffer is a Tris-EDTA buffer (TE buffer), which optionally is 1× through 5× TE buffer or 1× to 3× TE buffer or 1× TE buffer.

In some embodiments, the composition comprises, consists essentially of, or consists of the following excipients, in buffered solution, sodium sulfate, potassium sulfate, sodium chloride, potassium chloride, L-cysteine, and polysorbate-20, each optionally at concentrations already described (or about as described in Table 10 or Table 11). In certain of such embodiments the sulfate salts are at a concentration of about 2.5 mM to about 150 mM, or from about 10 mM to about 75 mM, or from about 25 mM to about 75 mM, or about 30 mM to about 75 mM. In certain embodiments the sodium sulfate is about 34 mM and the potassium sulfate is about 43 mM. In certain of such embodiments the chloride salts are each in a concentration of about 10 mM to about 1,000 mM, or from about 50 mM to about 600 mM, or from about 100 mM to about 500 mM, or is up to about 200 mM, or from about 100 mM to about 200 mM, or from about 136 mM to about 200 mM. In certain of such embodiments the sodium chloride is in a concentration of about 136 mM and the potassium chloride is in a concentration of about 136 mM. In certain of such embodiments the L-cysteine is at a concentration of about 0.1% w/v to about 1.5% w/v, or about 0.13% w/v. In some embodiments, the composition further comprises a cryoprotectant, which is optionally sucrose. In some embodiments, the buffer is TE buffer, which optionally is 1× through 5× TE buffer, or optionally 1× to 3× TE buffer.

In some embodiments, the composition comprises, consists essentially of, or consists of the following excipients, in buffered solution, potassium sulfate, sodium chloride, potassium chloride, L-methionine, and polysorbate-20, each optionally at concentrations already described (or about as described in Table 10 or Table 11). In certain of such embodiments the potassium sulfate is at a concentration of about 2.5 mM to about 150 mM, or from about 10 mM to about 75 mM, or up to about 75 mM, or from about 25 mM to about 75 mM, or about 30 mM to about 75 mM, or about 47 mM. In certain of such embodiments the chloride salts are each in a concentration of about 10 mM to about 1,000 mM, or from about 50 mM to about 600 mM, or from about 100 mM to about 500 mM, or is up to about 200 mM, or from about 100 mM to about 200 mM, or from about 136 mM to about 200 mM. In certain of such embodiments the sodium chloride is in a concentration of about 141 mM and the potassium chloride is in a concentration of about 180 mM. In certain such embodiments the methionine is about 0.1% w/v to about 1.5% w/v, or is about 0.15% w/v. In some embodiments, the composition further comprises a cryoprotectant, which is optionally sucrose. In some embodiments, the buffer is TE buffer, which is optionally 1× through 5× TE buffer, or optionally 1× to 3× TE buffer.

In some embodiments, the composition comprises, consists essentially of, or consists of the following excipients, in buffered solution, sodium sulfate, sodium chloride, L-cysteine, and polysorbate-20, each optionally at concentrations already described (or about as described in Table 10 or Table 11). In certain of such embodiments the sodium sulfate is at a concentration of about 2.5 mM to about 150 mM, or from about 10 mM to about 75 mM, or up to about 75 mM, or from about 25 mM to about 75 mM, or about 30 mM to about 75 mM, or about 48 mM. In certain of such embodiments the sodium chloride is in a concentration of up to about 200 mM, or from about 100 mM to about 200 mM, or from about 136 mM to about 200 mM, or about 192 mM. In certain of such embodiments the L-cysteine is at a concentration of about 0.1% w/v to about 1.5% w/v, or is about 0.1% w/v. In some embodiments, the composition further comprises a cryoprotectant, which is optionally sucrose. In some embodiments, the buffer is TE buffer, which is optionally 1× through 5× TE buffer, or optionally 1× to 3× TE buffer.

In some embodiments, the composition comprises, consists essentially of, or consists of the following excipients, in buffered solution, sodium sulfate, potassium sulfate, sodium chloride, potassium chloride, and polysorbate-20, each optionally at concentrations already described (or about as described in Table 10 or Table 11). In certain of such embodiments the sulfate salts are each at a concentration of about 2.5 mM to about 150 mM, or from about 10 mM to about 75 mM, or up to about 75 mM, or from about 25 mM to about 75 mM, or about 30 mM to about 75 mM, or about 75 mM. In certain of such embodiments the chloride salts are each in a concentration of about 10 mM to about 1,000 mM, or from about 50 mM to about 600 mM, or from about 100 mM to about 500 mM, or is up to about 200 mM, or from about 100 mM to about 200 mM, or from about 136 mM to about 200 mM, or about 200 mM. In certain of such embodiments the sodium chloride is in a concentration of about 136 mM and the potassium chloride is in a concentration of about 136 mM. In some embodiments, the composition further comprises a cryoprotectant, which is optionally sucrose. In some embodiments, the buffer is TE buffer, which is optionally 1× through 5× TE buffer, or optionally 1× to 3× TE buffer.

In some embodiments, the composition comprises, consists essentially of, or consists of the following excipients, in buffered solution, sodium sulfate, sodium chloride, potassium chloride, and polysorbate-20, each at concentrations already described (or about as described in Table 10 or Table 11). In certain of such embodiments the sodium sulfate is at a concentration of about 2.5 mM to about 150 mM, or from about 10 mM to about 75 mM, or up to about 75 mM, or from about 25 mM to about 75 mM, or about 30 mM to about 75 mM, or is about 75 mM. In certain of such embodiments the chloride salts are each in a concentration of up about 10 mM to about 1,000 mM, or from about 50 mM to about 600 mM, or from about 100 mM to about 500 mM, or is up to about 200 mM, such as from about 100 mM to about 200 mM, or from about 136 mM to about 200 mM. In certain of such embodiments the sodium chloride is in a concentration of about 200 mM and the potassium chloride is in a concentration of about 125 mM. In some embodiments, the composition further comprises a cryoprotectant, which is optionally sucrose. In some embodiments, the buffer is TE buffer, which is optionally 1× through 5× TE buffer, or optionally 1× to 3×TE buffer.

The LNP is a lipid delivery vehicle for the encapsulated mRNA. As known in the art, the LNPs for encapsulating mRNA may comprise: a cationic or ionizable lipid, a neutral lipid, a cholesterol or cholesterol moiety, and a PEGylated lipid. Lipid particle formulations that find use with embodiments of the present disclosure include those described in U.S. Pat. Nos. 9,738,593; 9,867,888, 10,221,127; 10,166,298; 10,266,485; and 10,442,756, which are each hereby incorporated by reference in their entireties. For example, the LNPs may comprise: an ionizable lipid; one or more selected from myristoylphosphatidylcholine (DMPC), dipalmitoylphosphatidylcholine (DPPC), and distearoylphosphatidylcholine (DSPC); cholesterol; and a PEG-lipid. In some embodiments, the LNPs comprise DSPC.

Non-limiting examples of PEG-lipids include PEG-modified phosphatidylethanolamine and phosphatidic acid, PEG-ceramide conjugates, PEG-modified dialkylamines and PEG-modified 1,2-diacyloxypropan-3-amines. In some embodiments, the PEG-lipid includes 1,2-dimyristoyl-sn-glycerol methoxypolyethylene glycol (PEG-DMG), 1,2-distearoyl-sn-glycero-3-phosphoethanolamine-N-[amino(polyethyleneglycol)] (PEG-DSPE), PEG-disteryl glycerol (PEG-DSG), PEG-dipalmetoleyl, PEG-diolcyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG-lipid is selected from a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

Suitable LNP compositions are further described in Table 1 of L. Shoenmaker et al., Int'l J. Pharmaceutics 601 (2021). For example, exemplary compositions can comprise DSPC, cholesterol, and the ionic lipid known as ALC-315 and the PEG-lipid known as ALC-0159. In other embodiments, the compositions can comprise DSPC, cholesterol, PEG-lipid, and the ionizable lipid known as SM-102. Other exemplary lipids and LNP compositions are described in U.S. Pat. No. 9,708,628 and US 2021/0023008, which are hereby incorporated by reference in their entireties. For example, LNPs can comprise disulfide linked lipids as the cationic or ionizable lipid, such as those known as SS-EC or SS-OP (under the trademark COATSOME). LNP compositions, including ratios of lipid components for producing encapsulated RNA for delivery to cells, are well known in the art.

For example, the cationic or ionizable lipid may be present from about 20 mol % to about 60 mol % of the lipids, such as from about 30 mol % to about 60 mol %, or from about 40 mol % to about 60 mol %. For example, the cationic or ionizable lipid may be present from about 45 mol % to about 55 mol % (or about 50 mol %). In some embodiments, the PEG-modified lipid is present at from about 0.5 mol % to about 10 mol %, such as from about 1mol % to about 5 mol %, or from about 1 mol % to about 2 mol %. In some embodiments, the neutral lipid (e.g. DSPC) is present in the LNP at from about 5 mol % to about 20 mol %, such as from about 5 mol % to about 15 mol % (or about 10 mol %). In various embodiments, cholesterol is present at from about 20 mol % to about 60 mol %, such as from about 30 mol % to about 50 mol %, or from about 35 mol % to about 45 mol %.

The RNA in various embodiments is included in the composition at a concentration of from about 0.01 to about 2.0 mg/mL, or from about 0.01 to about 1.0 mg/mL, or from about 0.05 to about 0.5 mg/mL, or about 0.1 mg/mL. The encapsulated RNA in various embodiments may be messenger RNA (mRNA). In other embodiments, the RNA may be an RNAi-inducing agent, such as an siRNAs, shRNAs, or miRNA. Alternatively, the RNA is an antisense oligonucleotide (such as an antisense oligonucleotide comprising RNA nucleotides), a ribozyme, or a gRNA. The RNA in some embodiments may be a self-amplifying RNA.

In various embodiments the RNA is a therapeutic or prophylactic RNA such as those described in PCT/US2022/02729 (WO 2022/32687) or US 2022/0370599, both of which are hereby incorporated by reference in their entirety.

In various embodiments, the RNA is messenger RNA (mRNA). In various embodiments, the mRNA is at least about 50 nucleotides in length, or is at least about 100 nucleotides in length, or is at least about 200 nucleotides in length, or is at least about 400 nucleotides in length, or is at least about 500 nucleotides in length, or is at least about 700 nucleotides in length, or is at least about 1000 nucleotides in length, or is at least about 1200 nucleotides in length, or at least about 1500 nucleotides in length, or at least about 2000 nucleotides in length, or at least 4000 nucleotides.

In some embodiments, the mRNA is transcribed from a DNA template in vitro or in a cell-free system, e.g., using T7 RNA polymerase. In vitro transcription is well known in the art. In some embodiments, the mRNA is synthesized using a cell-free process as described in WO 2020/205793 or U.S. Pat. No. 10,858,385, which are hereby incorporated by reference in their entireties, or as described herein. For example, in vitro transcription or cell-free transcription processes will involve a DNA template having a promoter. The DNA template will comprise an open reading frame (“ORF”) encoding the protein of interest and with untranslated regions. If positioned on the 5′ side of the ORF, the untranslated region is called a 5′ UTR. If positioned on the 3′ side of the ORF, the untranslated region is called a 3′ UTR.

In some embodiments, the RNA is an mRNA encoding a component of an infectious agent, such as a component of a virus, which is encapsulated with the LNPs to provide for an mRNA vaccine composition.

In some embodiments, the mRNA encodes one or more proteins of a virus or one or more polypeptides derived from virus proteins, for example, a DNA or RNA virus. Examples include those of the family Paramyxoviridae and/or genus Pneumovirinae or Morbillivirus. Example viruses include human metapneumovirus (hMPV), parainfluenza virus (hPIV), (types 1, 2, and 3), respiratory syncytial virus (RSV), and Measles virus (MeV). In some embodiments, the RNA virus is a coronavirus (CoV) (subfamily Coronavirinae, of the family Coronaviridae). In some embodiments, the coronavirus is a betacoronavirus, such as SARS-CoV or MERS-COV. In some embodiments, the RNA virus is SARS-COV-2, or a natural variant thereof. In other embodiments, the virus is a herpes virus, such as a herpes simplex virus or varicella zoster virus (e.g., gE antigen). In other embodiments, the virus is RSV, a hepatitis virus, or an adenovirus. In still other embodiments, the virus is an Ebola virus.

In some embodiments, the mRNA encodes one or more viral structural proteins or one or more polypeptides derived from virus proteins, such as a protein comprised in the viral envelop, such as a Spike protein(S) for coronaviruses. Alternatively or in addition, the mRNA encodes other CoV structural proteins such as M (membrane) glycoprotein, E (envelope) protein, and/or N (nucleocapsid) protein. Alternatively, an mRNA encoding the Spike protein or other structural protein can be encapsulated in particles that comprise or are decorated with one or more CoV structural proteins or portions thereof.

In some embodiments, the mRNA encodes one or more influenza proteins, such as neuraminidase (NA), hemagglutinin (HA), matrix protein 2 (M2), and/or nucleoprotein (NP).

In other embodiments, the mRNA encodes a therapeutic protein. In some embodiments, mRNA is targeted for expression in tissue or organs selected from liver (e.g., hepatocytes), skin (e.g., keratinocytes), skeletal muscle, endothelial cells, epithelial cells of various organs including the lungs, or hematopoietic or immune cells (e.g., T cells, B cells, or macrophages), for example. For example, the mRNA may be designed to encode polypeptides of interest selected from vaccine targets, enzymes (including metabolic enzymes or endonucleases such as Cas endonucleases), antibodies or antigen-binding fragments thereof or antibody mimetics (including nanobodies or single chain antibodies), secreted proteins or peptides (including cytokines, growth factors, or soluble receptors for the same), plasma membrane proteins, cytoplasmic or cytoskeletal proteins, intracellular membrane bound proteins, nuclear proteins, proteins associated with human disease (including proteins having loss-of-function or gain-of-function mutations associated with human disease). In some embodiments, the therapeutic protein includes one or more cancer-associated epitopes (e.g., one or more mutations associated with cancer, including neoantigens), which may find use in a cancer vaccine. An exemplary embodiment in which the mRNA encodes for an antibody, open reading frames encoding heavy and light chains can be expressed from different mRNA molecules.

In some aspects, the present disclosure provides a method for increasing the stability of an RNA by formulating the RNA as an LNP-encapsulated RNA according to this disclosure. Such compositions are stable for at least three months at 2-8° C., or for at least six months at 2-8° C., or for at least twelve months at 2-8° C. The compositions can therefore be stored and/or distributed at temperatures in the range of 2-8° C., providing substantial advantages over current mRNA compositions.

In some aspects, the present disclosure provides a method for expressing an mRNA in cells of a patient or subject. The method comprises administering a composition of the disclosure in said patient or subject. In such aspects, the present disclosure provides a use of the compositions disclosed herein for delivering a therapeutic RNA (including but not limited to therapeutic mRNA), for treatment or prevention of disease. The compositions can be used to vaccinate against viral infection, including for SARS-COV, influenza virus, or shingles, among others. In these aspects, the disclosure provides methods for expressing an mRNA in cells of an animal patient or subject, comprising administering the composition of the disclosure in said animal. Subjects include mammals, which include human patients and veterinary patients, as well as farm animals such as a pig or a cow. In other aspects, the subject is a bird, such as a chicken.

In some aspects, the disclosure provides a method for preventing or reducing the probability of a viral infection in a patient or a population, such as (but not limited to) SARS-CoV-2 infection. In these embodiments, the method comprises administering an mRNA vaccine of the present disclosure expressing one or more viral proteins, such as SARS-COV-2 Spike protein and/or other SARS-COV-2 structural protein as described herein. In some embodiments, the mRNA vaccine is administered as a single dose. In some embodiments, the mRNA vaccine is administered as multiple (e.g. two or three) doses, with a booster one, two, or three weeks after an initial dose. Periodic boosters can be administered as needed. In accordance with the various aspects, the present disclosure provides for simplified global distribution over currently available mRNA vaccines, since sub-zero conditions are not required for storage and distribution and/or because stability of the vaccine is improved.

In some embodiments of this aspect, the disclosure provides a method for expressing a therapeutic protein in a patient, comprising administering the mRNA composition described herein. For example, diseases, disorders, and/or conditions for treatment or prevention, include: autoimmune disorders (e.g., diabetes, lupus, multiple sclerosis, psoriasis, rheumatoid arthritis); inflammatory disorders (e.g., arthritis, pelvic inflammatory disease); infectious diseases (e.g., viral infections, bacterial infections, fungal infections, and sepsis); neurological disorders (e.g., Alzheimer's disease, Huntington's disease; autism; Duchenne muscular dystrophy); cardiovascular disorders (e.g., atherosclerosis, hypercholesterolemia, thrombosis, clotting disorders, angiogenic disorders such as macular degeneration); metabolic disorders and liver disorders (e.g., ornithine transcarbamylase deficiency); proliferative disorders (e.g., cancer, benign neoplasms); respiratory disorders (e.g., chronic obstructive pulmonary disease or idiopathic pulmonary fibrosis); digestive disorders (e.g., inflammatory bowel disease, ulcers); musculoskeletal disorders (e.g., fibromyalgia, arthritis); endocrine, metabolic, and nutritional disorders (e.g., diabetes, osteoporosis); urological disorders (e.g., renal disease); psychological disorders (e.g., depression, schizophrenia); skin disorders (e.g., wounds, eczema); and blood and lymphatic disorders (e.g., anemia, hemophilia).

In some embodiments, the mRNA may encode a transgene for expression in a subject, such as is described in PCT/US2021/042015 or US 2022/0096525, which are hereby incorporated by reference in their entireties.

Exemplary diseases characterized by dysfunctional or aberrant protein activity include cystic fibrosis, sickle cell anemia, epidermolysis bullosa, amyotrophic lateral sclerosis, and glucose-6-phosphate dehydrogenase deficiency. In various embodiments, the present disclosure provides a method for treating such conditions or diseases in a patient by introducing an mRNA encoding for a protein that overcomes the aberrant protein activity present in the cell of the patient. Specific examples of a dysfunctional protein are the missense mutation variants of the cystic fibrosis transmembrane conductance regulator (CFTR) gene, which produce a dysfunctional protein variant of CFTR protein, which causes cystic fibrosis.

Other diseases characterized by missing or substantially diminished protein activity (such that proper, normal or physiological protein function does not occur) include cystic fibrosis, Niemann-Pick type C, B thalassemia major, Duchenne muscular dystrophy, Hurler Syndrome, Hunter Syndrome, and Hemophilia A. Such proteins may not be present, or are essentially non-functional. The present invention provides a method for treating such conditions or diseases in a patient by introducing mRNA provided herein (using the stable LNP-encapsulated RNA compositions described herein), wherein the mRNA encodes for a protein that replaces the protein activity missing from the target cells of the patient.

In various embodiments, the compositions are administered by parenteral administration, such as intramuscular, intradermal, subcutaneous, intravenous, or intrathecal administration. In other embodiments, the compositions (e.g., mRNA vaccines) described herein are administered intranasally.

As used herein, the term “about” means±10% of an associated numerical value unless the context requires otherwise.

As used herein, the word “include,” and its variants, is intended to be non-limiting, such that recitation of items in a list is not to the exclusion of other like items that may also be useful in the compositions and methods of this technology.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.” Unless otherwise stated, the transitional term “consisting essentially of” means that the composition can have other components that do not reduce the stability of LNP-encapsulated RNA as compared to a reference composition.

Other aspects and embodiments of the invention will be apparent from the following Examples.

### EXAMPLES

### Example 1

### Method/Materials

Lipids (ionizable lipid, DSPC, cholesterol, and PEG lipid) described in Table 1 (Col. 3) of L. Shoenmaker et al., Int'l J. Pharmaceutics 601 (2021) at similar ratio to that disclosed in the same, were dissolved in ethanol. The lipid mixture was combined with buffer using a microfluidic mixer (Precision Nanosystems, Vancouver, BC, Canada). Formulations were diluted with phosphate buffered saline (PBS), concentrated using Amicon ultra-centrifugal filters (EMD Millipore, USA), and then passed through a 0.22-μm filter and stored at 4° C. until use. Formulations were tested for particle size, polydispersity index, mRNA encapsulation, mRNA integrity, potency (% cells transfected) and mRNA expression. Particle size and polydispersity index were measured using the technique of dynamic light scattering. mRNA encapsulation was measured using commercially available RNA Assay kits (Quanti-iT™ RiboGreen™ RNA Assay Kit). mRNA integrity was measured using high performance liquid chromatography. Luciferase expression was tested on 293 FT cells. Both Covid-19 mRNA (SARS-COV-2 spike protein mRNA) and Luciferase mRNA were used in the studies.

The excipients selected for study are summarized in Table 1.

TABLE 1
Excipients

| | Proposed | Proposed | Initial |
|---|---|---|---|
| Excipient | Action | Purpose | concentration |
| Disodium | Metal ion | Prevent enzymatic | 0.1 mM |
| EDTA | chelator | degradation | |
| PBS buffer | Buffer | Prevent LNP | QS(quantity |
| (pH 7.4) | | aggregation | sufficient) |
| Sucrose | Reduce | Reduce mRNA | 15% w/v |
| | mRNA | degradation due to | |
| | exposure | water | |
| | to water | | |
| Ethanol | Scavenge | Prevent mRNA | 250 mM |
| | hydroxyl | degradation due to | |
| | radical | free radical oxidation | |
| Histidine | Scavenge | Prevent mRNA | 0.1% w/v |
| | hydroxyl | degradation due to | |
| | radical | free radical oxidation | |
| Polysorbate-20 | Non-ionic | Prevent surface | 0.01% w/v |
| | surfactant | adsorption | |
| Methionine, | Antioxidant | Prevent oxidation | 0.05, 0.1, |
| Ascorbic Acid, | | | 0.15% w/v |
| Citric Acid | | | |
| Sorbitol, | Stabilizing | Stabilizing LNPs | 10% w/v Sorbitol |
| Gelatin, | agent | | 10% w/v Gelatin |
| Glycine | | | 1.5% w/v Glycine |

### Results

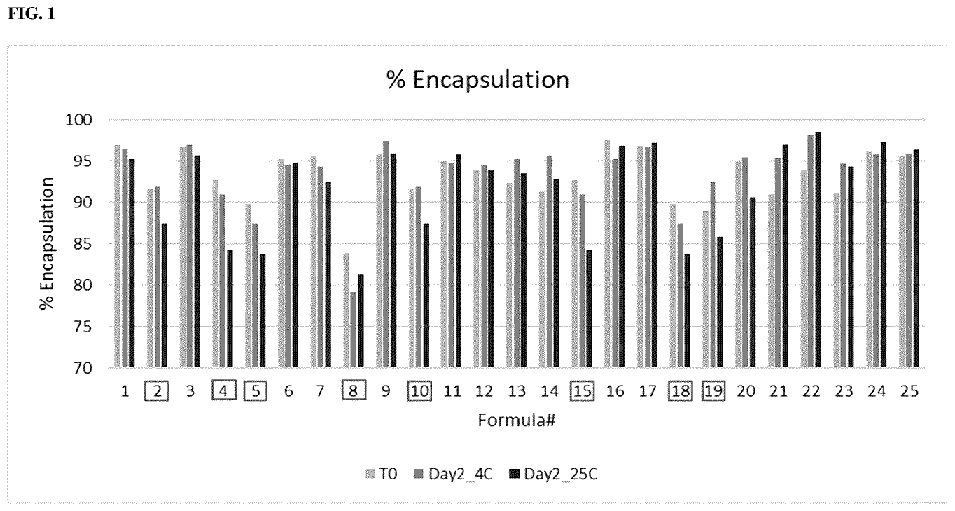

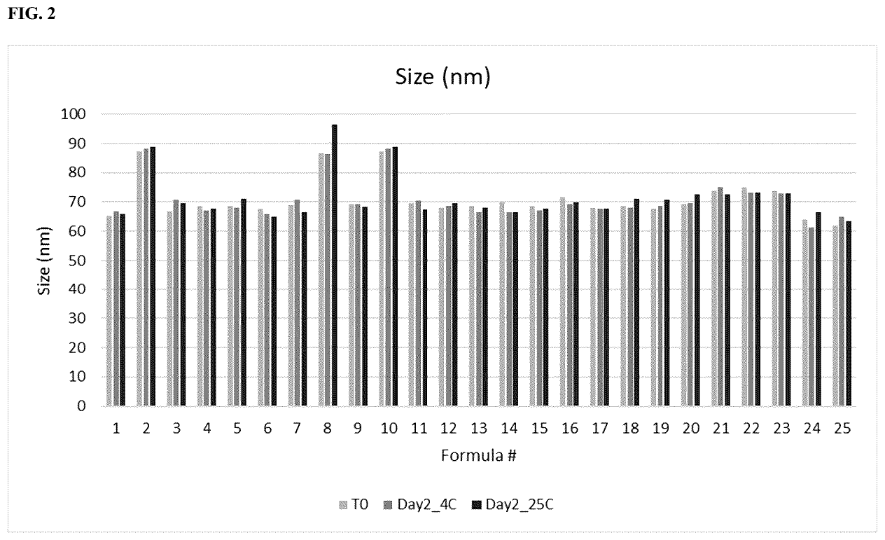

Before trying different permutations and combinations of the selected excipients (Table 1), LNP-encapsulated mRNAs were incubated (at both 4° C. and 25° C.) with individual excipients and LNP particle size, polydispersity index (PDI), and mRNA encapsulation were measured. Excipients that had an effect on size and/or encapsulation were ethanol, citric acid, ascorbic acid (FIG. 1, FIG. 2, Table 2).

TABLE 2
Initial Formulations Tested

| Formulation | Contents |
|---|---|
| 1 | LNP, PBS |
| 2 | LNP, Ethanol 250 mM, PBS |
| 3 | LNP, Sucrose, PBS |
| 4 | LNP, Ascorbic Acid 0.15% w/v, PBS |
| 5 | LNP, Citric acid 0.15% w/v, PBS |
| 6 | LNP, Methionine, PBS |
| 7 | LNP, Sorbitol, PBS |
| 8 | LNP, Ethanol, Sucrose, Ascorbic acid, Citric acid, |
| | Methionine, Sorbitol, PBS |
| 9 | LNP, EDTA 0.1 mM, PBS |
| 10 | LNP, Ethanol 250 mM, PBS |
| 11 | LNP, Ethanol 200 mM, PBS |
| 12 | LNP, Ethanol 150 mM, PBS |
| 13 | LNP, Ethanol 100 mM, PBS |
| 14 | LNP, Ethanol 50 mM, PBS |
| 15 | LNP, Ascorbic acid 0.15% w/v, PBS |
| 16 | LNP, Ascorbic acid 0.10% w/v, PBS |
| 17 | LNP, Ascorbic acid 0.05% w/v, PBS |
| 18 | LNP, Citric acid 0.15% w/v, PBS |
| 19 | LNP, Citric acid 0.10% w/v, PBS |
| 20 | LNP, Citric acid 0.05% w/v, PBS |
| 21 | LNP, Histidine 0.1% w/v, PBS |
| 22 | LNP, Glycine 1.5% w/v, PBS |
| 23 | LNP, Polysorbate-20 (PS-20) 0.01% w/v, PBS |
| 24 | LNP, PBS, Gelatin 5% w/v |
| 25 | LNP, PBS, Gelatin 10% w/v |

Ethanol at 250 mM resulted in increased LNP size. On reducing the ethanol concentration, LNP size was not affected (when compared to initial size). Therefore, ethanol concentration of 150 mM was selected for further evaluation. Encapsulation dropped in the presence of citric acid at 0.15% w/v and 0.1% w/v, and ascorbic acid 0.15% w/v. It was determined that the presence of citric acid or ascorbic acid reduced the pH of the system. Citric acid is a stronger acid than ascorbic and could have a greater effect on percent encapsulation over time. Citric acid was therefore eliminated from further evaluation.

The shortlisted excipients were used to set a custom experimental design. The excipients and drug product were selected as factors, and responses tested were LNP integrity (LNP particle size, polydispersity index, and mRNA encapsulation), and mRNA integrity. The mRNA concentration for these experiments was 0.1 mg/mL in PBS buffer (pH 7.4). The study summarized in Table 3 was conducted at 2° to 8° C. (long-term) and 25° C. (accelerated).

TABLE 3
Experiment 1 (conducted at 2° C to 8° C for long term and 25° C for accelerated study)

| | | | | Disodium | | | | |
|---|---|---|---|---|---|---|---|---|
| | PS-20 | | Antiox. | EDTA | Sucrose | Histidine | Ethanol | |
| Sample | (% w/v) | Stab. agent | (% w/v) | (mM) | (% w/v) | (% w/v) | (mM) | Antioxidant |
| 1 | 0.01 | Sorbitol | 0.15 | 0 | 15 | 0 | 150 | Methionine |
| 2 | 0.01 | Sorbitol | 0.05 | 0.1 | 0 | 0.1 | 0 | Ascorbic acid |
| 3 | 0 | Gelatin | 0.15 | 0 | 0 | 0.1 | 0 | Methionine |
| 4 | 0 | Gelatin | 0.05 | 0.1 | 15 | 0.1 | 150 | Ascorbic acid |
| 5 | 0 | Glycine | 0.15 | 0.1 | 0 | 0 | 150 | Methionine |
| 6 | 0 | Glycine | 0.05 | 0 | 15 | 0 | 0 | Ascorbic acid |
| 7 | 0 | Sorbitol | 0.05 | 0 | 0 | 0 | 150 | Methionine |
| 8 | 0 | Sorbitol | 0.15 | 0.1 | 15 | 0.1 | 0 | Ascorbic acid |
| 9 | 0.01 | Gelatin | 0.05 | 0.1 | 15 | 0 | 0 | Methionine |
| 10 | 0.01 | Gelatin | 0.15 | 0 | 0 | 0 | 150 | Ascorbic acid |
| 11 | 0.01 | Glycine | 0.05 | 0 | 15 | 0.1 | 150 | Methionine |
| 12 | 0 | 0 | 0 | 0 | 0 | 0 | 0 | 0 |

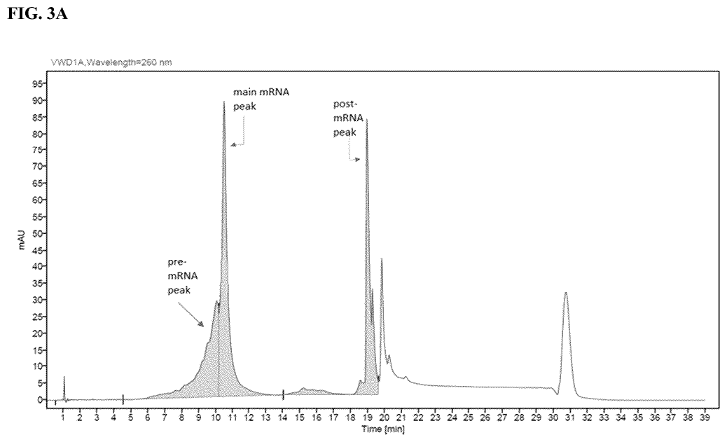

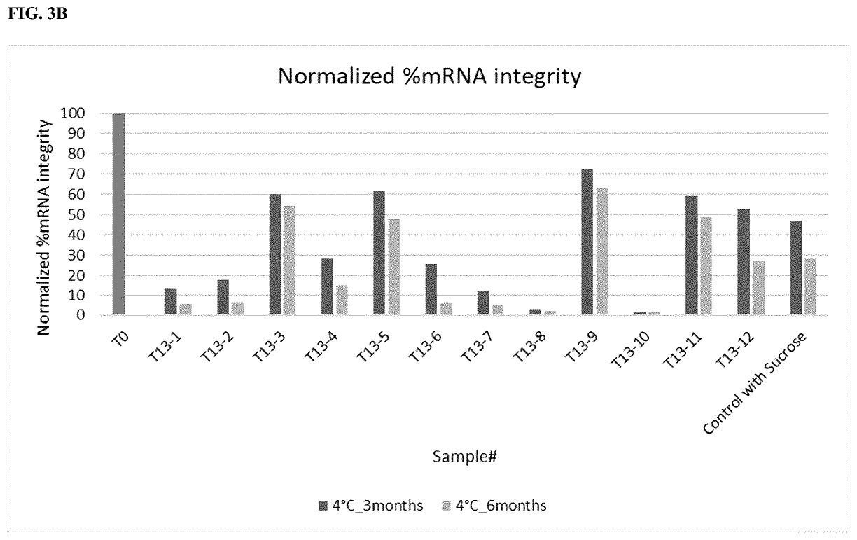

After three months at 4° C., mRNA integrity was measured via HPLC. Degraded mRNA is seen in the resultant chromatogram (FIG. 3A) in the pre-mRNA peak of degraded mRNA that eluded before main intact mRNA peak, and the post-mRNA peak of degraded mRNA that eluded only after the main mRNA peak. To calculate mRNA integrity, only the main mRNA peak was considered as intact mRNA, as shown in FIG. 3A. (See, e.g., Packer et al., A novel mechanism for the loss of mRNA activity in lipid nanoparticle delivery systems. Nature Communications vol. 12, Article 6777 (2021)). The mRNA integrity was normalized with respect to Day 0 and results are shown in FIG. 3B.

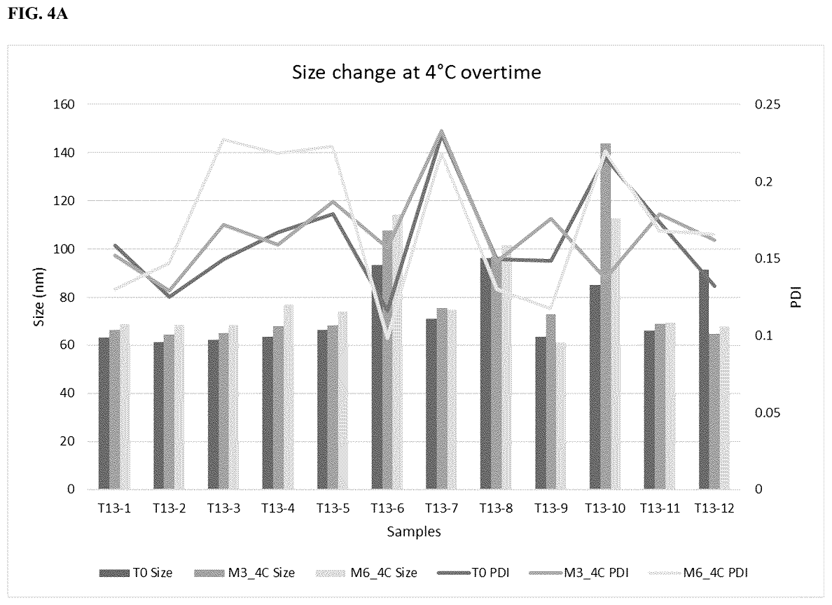

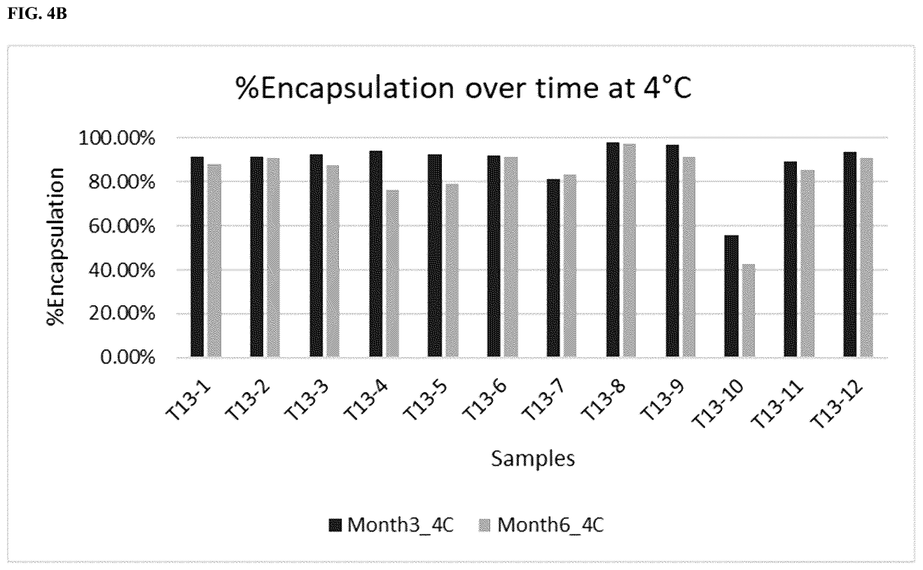

From the LNP size (FIG. 4A), mRNA encapsulation (FIG. 4B) and mRNA integrity data (FIG. 3B), only a few samples performed better than the controls. Samples T13-3, T13-5, T13-9 and T13-11 were selected for further experimental design. The following excipients were used in samples T13-3, T13-5, T13-9 and T13-11:
1) L-Methionine (0.15% w/v or 0.05% w/v),
2) Hydrolyzed Gelatin (10% w/v),
3) Glycine (1.5% w/v),
4) Disodium EDTA (0.1% w/v),
5) Sucrose (15% w/v),
6) Histidine (0.1% w/v),
7) Polysorbate-20 (0.01% w/v), and
8) Ethanol (150 mM).

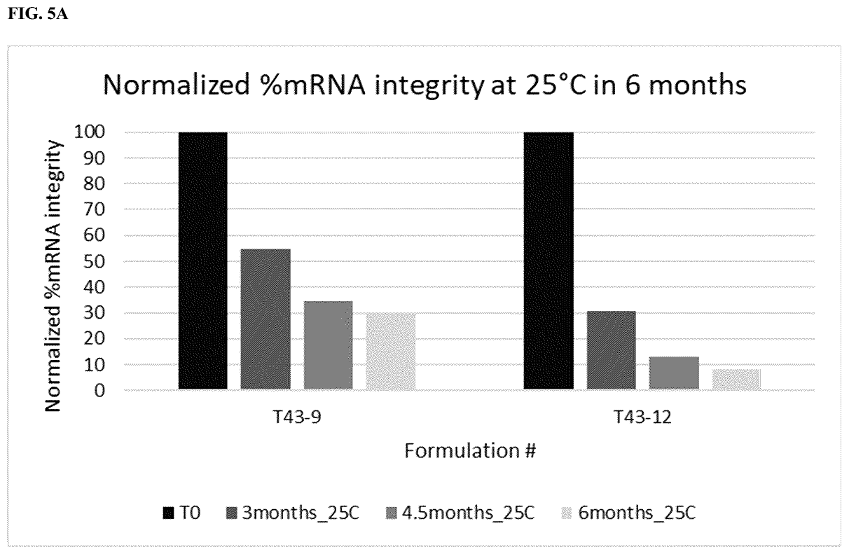

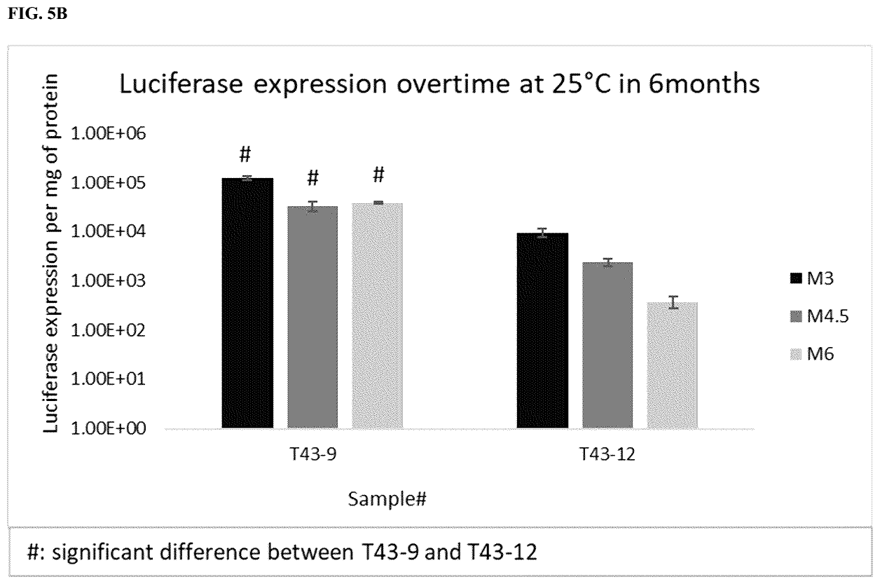

Sample T13-9 [0.01% w/v polysorbate-20, 10% w/v gelatin, 0.05% w/v L-methionine, 0.1 mM disodium EDTA, 15% w/v sucrose, and 0.1 mg/mL LNP-encapsulated mRNA in PBS buffer (pH 7.4)] was particularly promising, and therefore a repeat study with Sample T13-9 and Sample T13-12 (as control; LNP-encapsulated mRNA in PBS) was conducted. The study was conducted at −20° C. (frozen condition), 4° C. (long-term), and 25° C. (accelerated conditions) at mRNA concentration of 0.1 mg/mL, using an approximately 1900 nucleotide length mRNA coding for luciferase. Accelerated testing at room temperature (25° C.) was conducted for the purpose of predicting stability profile in a shorter period. In six months at 25° C., Sample T43-9 (repeat of T13-9) had approximately 4-fold better mRNA integrity than the control (Sample T43-12, which is repeat of T13-12) (FIG. 5A), which confirms the behavior observed in Experiment 1. Sample T43-9 further demonstrated improved protein expression (luciferase) compared to control (T43-12) after six months at 25° C. (approximately 100-fold difference) (FIG. 5B).

From the selected excipients from Experiment 1, a further experimental design was constructed. The excipients and drug product (LNP; DP) were selected as factors, and responses tested were LNP integrity (LNP particle size, PDI, and mRNA encapsulation), mRNA integrity, and luciferase expression. The study was designed to study the following interactions:
1) Methionine*Histidine
2) Methionine*Sucrose
3) Methionine*Disodium EDTA
4) Methionine*Stabilizing agent
5) Histidine*Sucrose
6) Histidine*Disodium EDTA
7) Histidine*Stabilizing agent
8) Sucrose*Disodium EDTA
9) Sucrose*Stabilizing agent
10) Disodium EDTA*Stabilizing agent

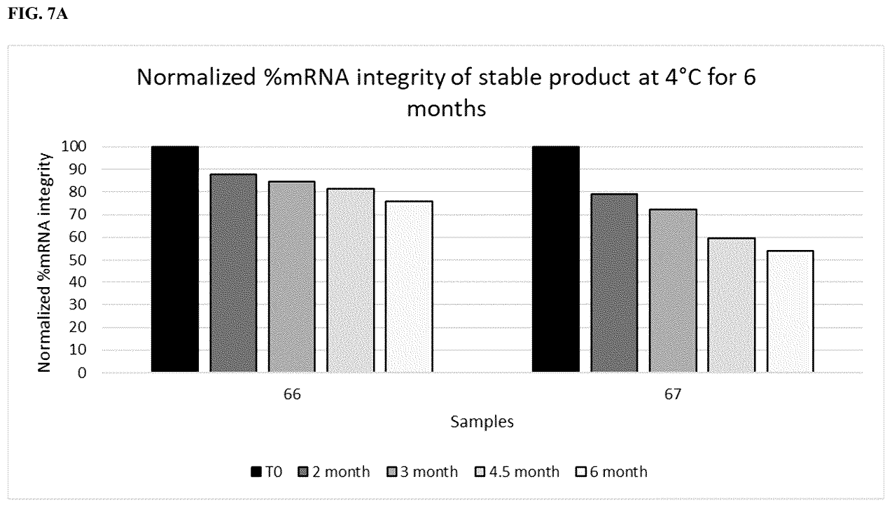

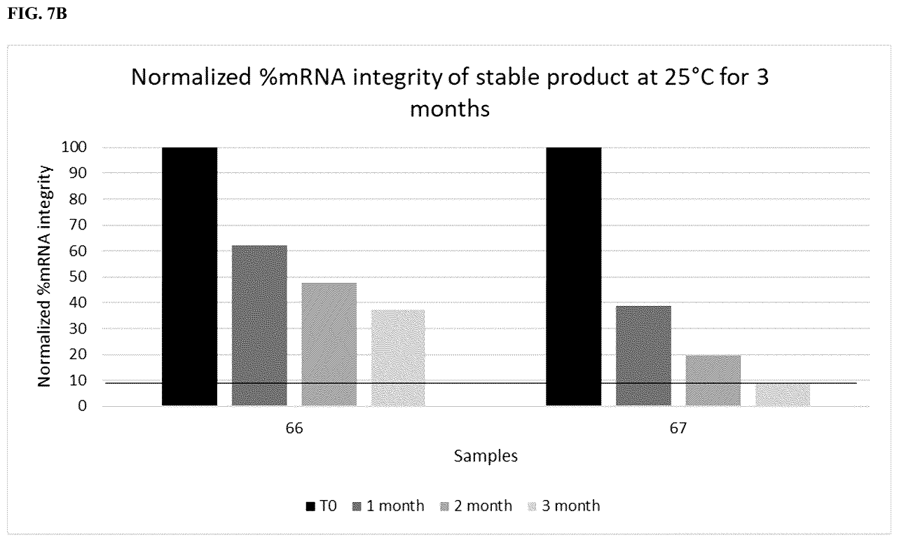

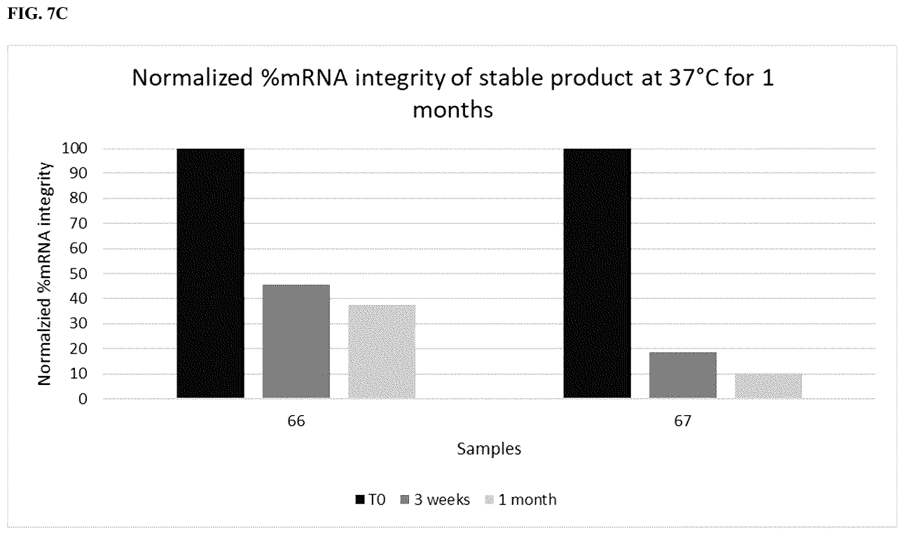

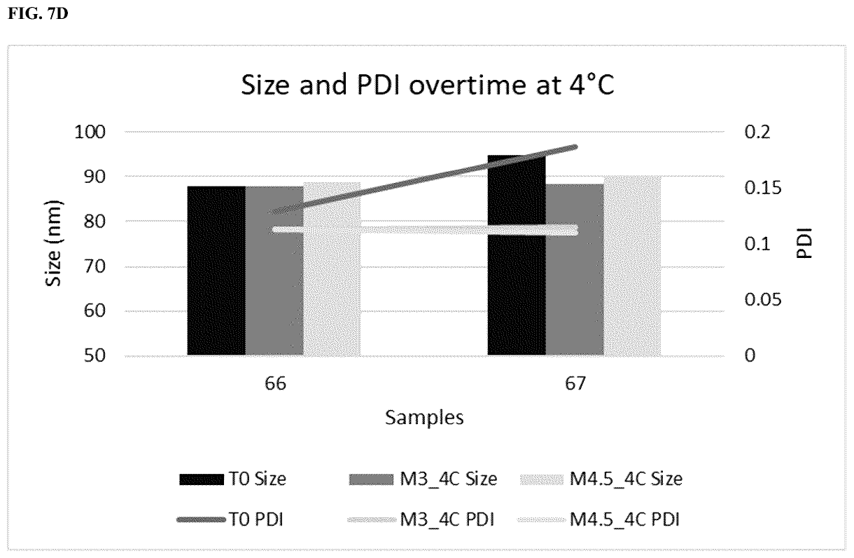

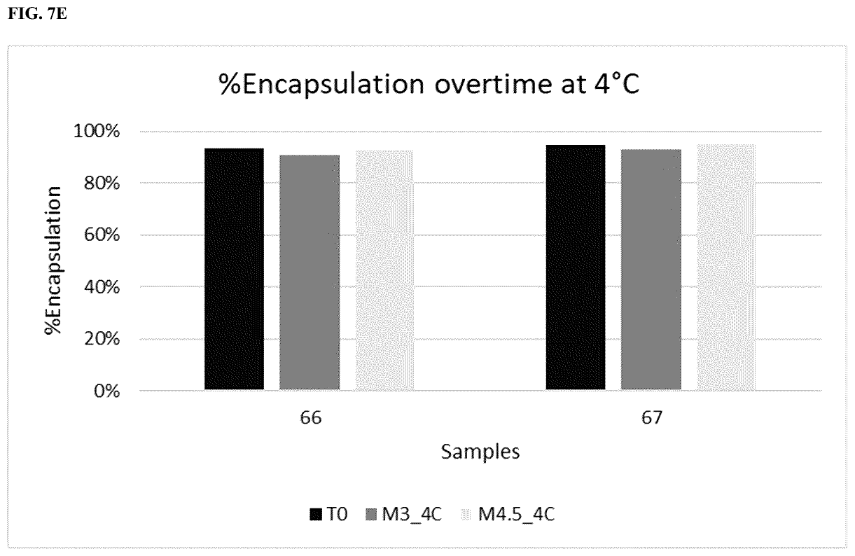

Two sample compositions (duplicates) were locked in as benchmarks (#1 and #12) which represented Sample T13-9 of Experiment 1 and one control (C, #20), which represented Sample T13-12 of Experiment 1. The resulting experimental rubric consisted of 25 runs or samples (Table 4). The study was conducted at 37° C. (accelerated conditions) at mRNA concentration of 0.1 mg/mL. Methionine, Polysorbate-20, and Glycine were identified as likely having significant contributions towards maximizing mRNA integrity and luciferase expression. From the results of Experiment 2, the best performing formulation was determined to be: 0.15% w/v methionine, 0.01% w/v polysorbate-20, 1.5% w/v glycine, in buffered solution (e.g., PBS). This formulation with the addition of 0.1 mM EDTA and PBS buffer at 7.4 pH and mRNA concentration of 0.1 mg/ml (Formulation 66) was tested for 6 months at 4° C. (FIG. 7A), 3 months at 25° C. (FIG. 7B), and 1 month at 37° C. (FIG. 7C). The LNP-encapsulated mRNA was approximately 4000 nucleotides in length and contained a coding region for SARS-COV-2 spike protein. A solution with 10% Sucrose in PBS with the same LNP encapsulated mRNA as the test sample was used as a control (Formulation 67). The test sample was superior at all temperatures and time periods. It was nearly 4-fold better than control at 37° C. after 1 month and 25° C. after 3 months. No change in size, PDI or % encapsulation (from Day0) observed overtime at 4° C. (FIGS. 7D, 7E). Hence formulation 66 provides better stability than the control, maintaining stability for at least 6 months at 4° C.

TABLE 4
Experiment 2 (conducted at 37° C for accelerated study)

| | | | | | Disodium | | | Stabilizing |
|---|---|---|---|---|---|---|---|---|
| Sample | Methionine | Histidine | Sucrose | PS-20 | EDTA | DPBS | DP | Agent |
| 1 | 0.15% | 0.00% | 15.00% | 0.01% | 0.10% | QS | 0.10% | Gelatin |
| | | | | | | (84.64%) | | |
| 2 | 0.00% | 0.00% | 0.00% | 0.01% | 0.10% | QS | 0.10% | Gelatin |
| | | | | | | (99.79%) | | |
| 3 | 0.00% | 0.10% | 15.00% | 0.01% | 0.00% | QS | 0.10% | Glycine |
| | | | | | | (84.79%) | | |
| 4 | 0.15% | 0.00% | 0.00% | 0.00% | 0.10% | QS | 0.10% | None |
| | | | | | | (99.65%) | | |
| 5 | 0.15% | 0.00% | 0.00% | 0.01% | 0.10% | QS | 0.10% | Glycine |
| | | | | | | (99.64%) | | |
| 6 | 0.00% | 0.10% | 15.00% | 0.00% | 0.00% | QS | 0.10% | None |
| | | | | | | (84.80%) | | |
| 7 | 0.15% | 0.00% | 15.00% | 0.00% | 0.00% | QS | 0.10% | Glycine |
| | | | | | | (84.75%) | | |
| 8 | 0.00% | 0.00% | 15.00% | 0.01% | 0.10% | QS | 0.10% | None |
| | | | | | | (84.79%) | | |
| 9 | 0.15% | 0.00% | 15.00% | 0.01% | 0.00% | QS | 0.10% | None |
| | | | | | | (84.74%) | | |
| 10 | 0.00% | 0.10% | 0.00% | 0.00% | 0.10% | QS | 0.10% | Glycine |
| | | | | | | (99.70%) | | |
| 11 | 0.15% | 0.10% | 0.00% | 0.01% | 0.10% | QS | 0.10% | Gelatin |
| | | | | | | (99.54%) | | |
| 12 | 0.15% | 0.00% | 15.00% | 0.01% | 0.10% | QS | 0.10% | Gelatin |
| | | | | | | (84.64%) | | |
| 13 | 0.00% | 0.00% | 15.00% | 0.00% | 0.10% | QS | 0.10% | Glycine |
| | | | | | | (84.80%) | | |
| 14 | 0.15% | 0.00% | 0.00% | 0.00% | 0.00% | QS | 0.10% | Gelatin |
| | | | | | | (99.75%) | | |
| 15 | 0.15% | 0.10% | 15.00% | 0.01% | 0.00% | QS | 0.10% | Gelatin |
| | | | | | | (84.64%) | | |
| 16 | 0.15% | 0.10% | 0.00% | 0.01% | 0.00% | QS | 0.10% | None |
| | | | | | | (99.64%) | | |
| 17 | 0.00% | 0.10% | 0.00% | 0.01% | 0.00% | QS | 0.10% | Gelatin |
| | | | | | | (99.79%) | | |
| 18 | 0.00% | 0.00% | 0.00% | 0.01% | 0.00% | QS | 0.10% | Glycine |
| | | | | | | (99.89%) | | |
| 19 | 0.00% | 0.10% | 0.00% | 0.01% | 0.10% | QS | 0.10% | None |
| | | | | | | (99.69%) | | |
| 20 (C) | 0.00% | 0.00% | 0.00% | 0.00% | 0.00% | QS | 0.10% | None |
| | | | | | | (99.90%) | | |
| 21 | 0.15% | 0.10% | 15.00% | 0.00% | 0.10% | QS | 0.10% | None |
| | | | | | | (84.55%) | | |
| 22 | 0.15% | 0.10% | 0.00% | 0.00% | 0.00% | QS | 0.10% | Glycine |
| | | | | | | (99.65%) | | |
| 23 | 0.00% | 0.00% | 15.00% | 0.01% | 0.00% | QS | 0.10% | Gelatin |
| | | | | | | (84.89%) | | |
| 24 | 0.00% | 0.10% | 15.00% | 0.00% | 0.10% | QS | 0.10% | Gelatin |
| | | | | | | (84.70%) | | |
| 25 | 0.15% | 0.10% | 15.00% | 0.01% | 0.10% | QS | 0.10% | Glycine |
| | | | | | | (84.54%) | | |

### Conclusion

From the results of Experiment 1, it was determined that six months stability of LNP-encapsulated mRNA at 4° C. (2° C. to 8° C.) may be feasible by selection of excipients. Experiments identified the excipients methionine, polysorbate-20, and glycine as having a significant contribution towards maximizing the mRNA integrity and luciferase expression.

### Example 2

### Method/Materials

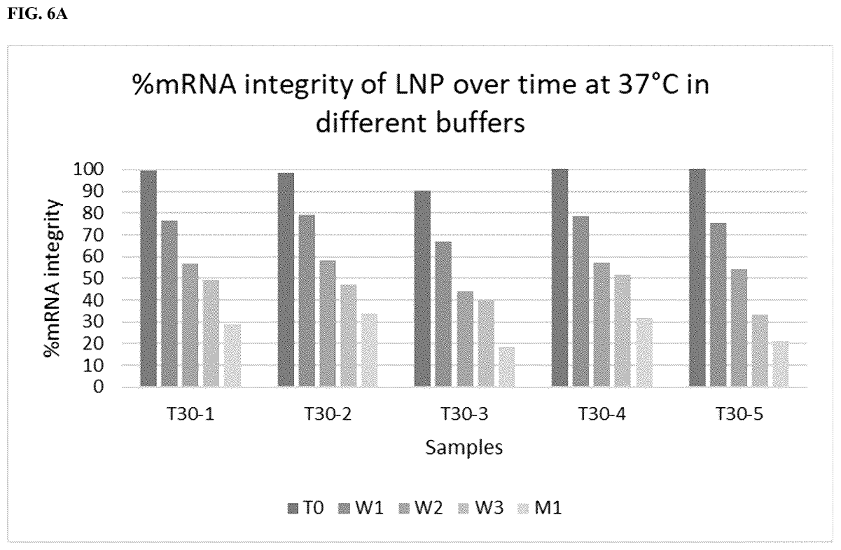

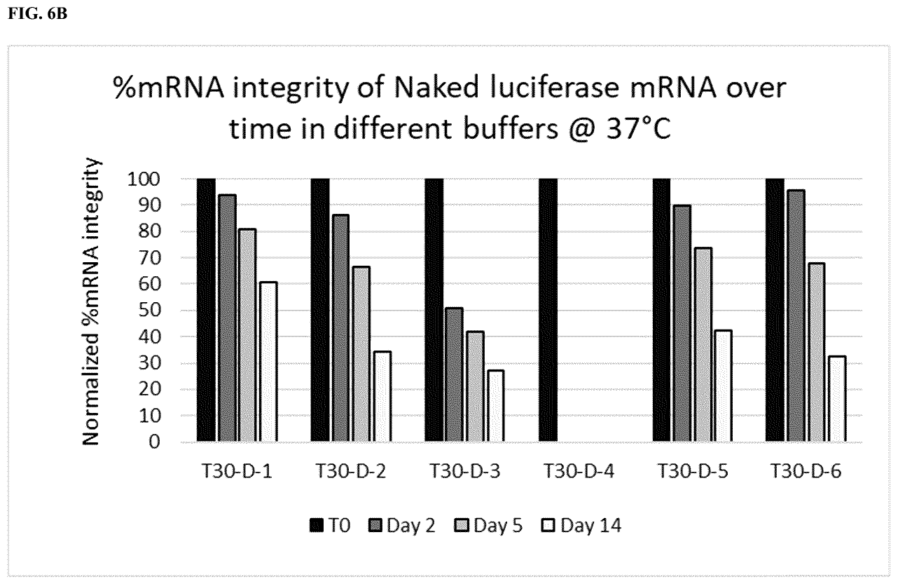

Concentrated LNP-encapsulated mRNA (encoding luciferase) was diluted with various buffers to an mRNA concentration of 0.1 mg/mL. Samples were kept at 37° C. for one month. This testing was conducted at elevated temperature, here body temperature, for purposes of predicting stability profile in a shorter period. Aliquots were pulled after every week and mRNA integrity was tested. Results are shown in FIG. 6A, demonstrating that histidine buffer (T30-1), TE buffer (T30-2), and Na citrate buffer (T30-4) provided better stability over 1 month at 37° C. than Tris acetate buffer (T30-3) and PBS buffer (T30-5).

TABLE 5
Buffer Evaluation

| Sample | Buffer |
|---|---|
| T30-1 | Histidine Buffer (L-Histidine, pH 8.0) |
| T30-2 | Tris-EDTA (TE) (Tris HCl, Disodium EDTA, pH 7.5) |
| T30-3 | Tris-Acetate (Tris base, Sodium Acetate, pH 7.5) |
| T30-4 | Na Citrate (Sodium Citrate Dihydrate, Citric Acid, pH 6.4) |
| T30-5 | PBS |

Similar buffers were tested with naked mRNA diluted to 0.1 mg/mL, kept at 37° C. for five (5) days and mRNA integrity was tested. Results are shown in FIG. 6B, showing that TE buffer provided the best stability to naked mRNA (T30-D-1) followed by sodium citrate buffer (T30-D-5) followed by PBS (T30-D-6) and histidine buffer (T30-D-2).

TABLE 6
Buffers evaluated with naked mRNA

| Sample | Buffer |
|---|---|
| T30-D-1 | TE Buffer pH 7.5 |
| T30-D-2 | Histidine Buffer pH 8.0 |
| T30-D-3 | Tris-Acetate Buffer pH 7.5 |
| T30-D-4 | Citrate Buffer pH 4.0 |
| T30-D-5 | Sodium Citrate Buffer pH 6.4 |
| T30-D-6 | PBS |

From both the LNP and naked mRNA stability results (FIGS. 6A and 6B), TE buffer was selected as most beneficial for stability amongst the other buffers tested.

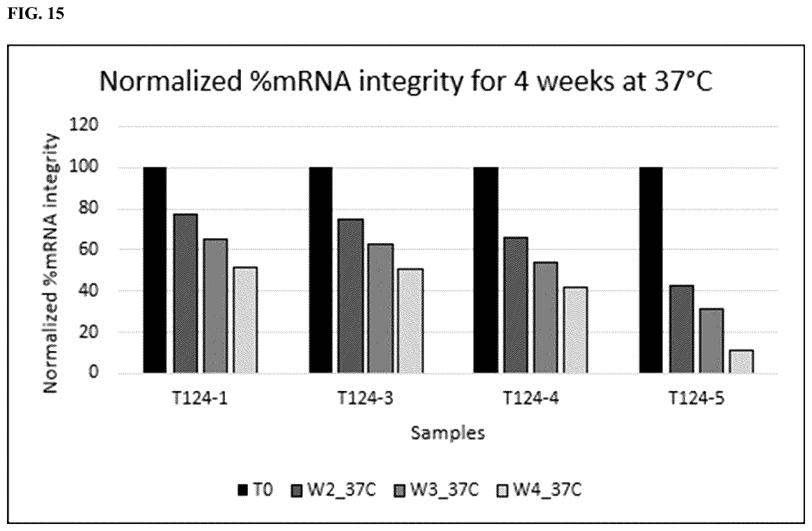

TE Buffer was further evaluated at concentrations of 1×, 3×, 5×, and 10× as set forth in Table 13. mRNA integrity was evaluated for encapsulated luciferase mRNA in TE buffer over 4 weeks at 37° C. Results are shown in FIG. 15 and indicate that concentrations of 1× and 3× provided for similar stability over the entire four weeks.

TABLE 13
TE Buffers Evaluated with Encapsulated mRNA

| | Buffer used to make and dilute |
|---|---|
| Samples | LNP |
| T124-1 | 1X TE Buffer |
| T124-3 | 3X TE Buffer |
| T124-4 | 5X TE Buffer |
| T124-5 | 10X TE Buffer |

### Example 3

Lipids (ionizable lipid, DSPC, cholesterol, and PEG lipid) described in Table 1 (Col. 3) of L. Shoenmaker et al., Int'l J. Pharmaceutics 601 (2021), at similar ratio to those described therein, were dissolved in ethanol. Formulations were diluted with phosphate buffered saline (PBS) or TE buffer (10 mM Tris HCl and 1 mM Disodium EDTA) pH 7.5 and concentrated using Amicon ultra-centrifugal filters (EMD Millipore, USA) and then passed through a 0.22-μm filter and stored at 4° C. until use. Formulations were tested for particle size, polydispersity index, mRNA encapsulation, mRNA integrity, potency (% cells transfected) or mRNA expression. Particle size and polydispersity index were measured using technique of dynamic light scattering. mRNA encapsulation was measured using commercially available RNA Assay kits. mRNA integrity was measured using high performance liquid chromatography (HPLC), as described in Example 1 (except for testing of naked mRNA where capillary electrophoresis is specifically identified). Luciferase expression was tested on 293 FT cells. Both Covid-19 mRNA (SARS-COV-2 spike protein mRNA) and Luciferase mRNA were used in the studies. To test the ability of antioxidants, ionizable lipid was subjected to Fenton reaction where it underwent forceful oxidation, and different antioxidant levels were tested to obtain a range that provides best protection against oxidation. Ionizable lipid dissolved in ethanol was mixed with iron sulfate and antioxidant (if any) dissolved in water along with hydrogen peroxide and incubated at 37° C. overnight. Ionizable lipid was separated and analyzed using HPLC.

Table 7 lists certain excipients that may be employed in various combinations according to embodiments of the present disclosure.

TABLE 7

| Category | Excipient |
|---|---|
| Buffer | 1X TE, PBS, Histidine, Sodium acetate, |
| | Sodium citrate |
| Non-ionic | Polysorbate-20, Polysorbate-60, Polysorbate-80 |
| surfactant | |
| Antioxidant | Methionine, Cysteine |
| Salts | Sodium Chloride, Potassium Chloride, |
| | Sodium Sulfate, Potassium Sulfate |
| Sugars/ | Sucrose |
| Cryoprotectant | |

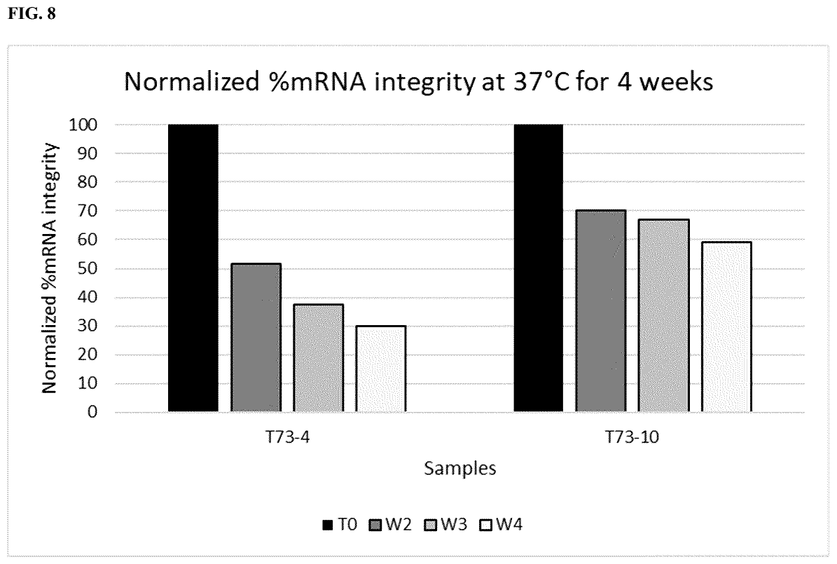

To determine the desired buffer, LNP-encapsulated Luciferase mRNA at 0.1 mg/mL with sucrose (10% w/v) and either PBS buffer (sample T73-4) or 1× TE (sample T73-10) was tested at 37° C. for 4 weeks and % mRNA integrity was measured. Results are shown in FIG. 8. TE had a significantly greater effect on mRNA stability than PBS maintaining 70% normalized integrity after 2 weeks at 37° C. and maintaining nearly 60% normalized integrity after 4 weeks at 37° C.

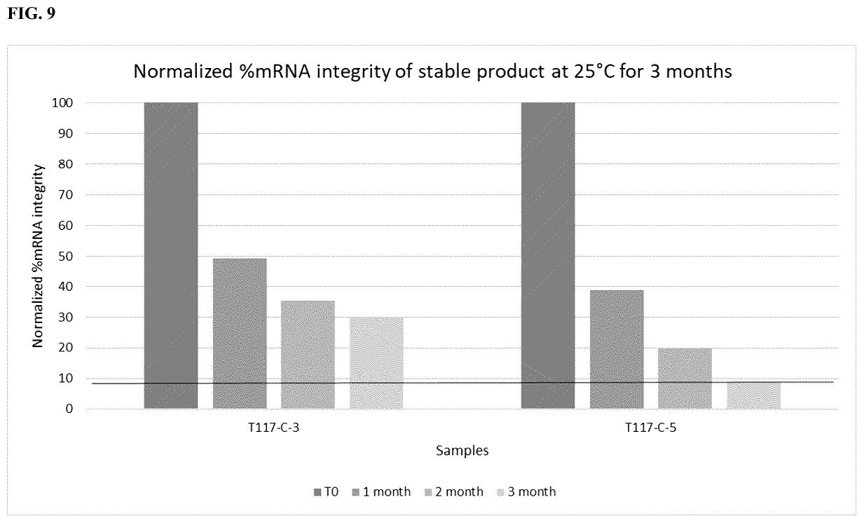

The effect of Polysorbate-20 on the formulation was further investigated by comparing two LNP-encapsulated SARS-COV-2 spike protein mRNA at 0.1 mg/ml formulations: a control (T117-c-5) with sucrose (15% w/v) in PBS buffer; and a test sample T117-c-3 with sucrose (15% w/v), 0.01% w/v Polysorbate-20, and 0.1 mM EDTA in PBS buffer. Both formulations were tested at 25° C. for 3 months and % mRNA integrity was measured at start, 1 month, 2, month, and 3 months. Results are shown in FIG. 9 demonstrating the positive effect of Polysorbate-20 on mRNA integrity. As shown in FIG. 9 polysorbate-20 provides increased stability of mRNA versus the control.

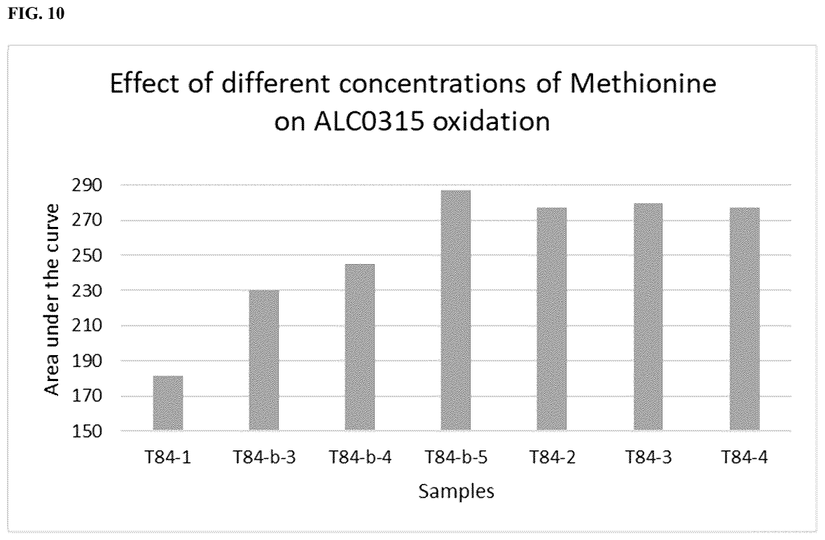

The effect of Methionine on the formulations was tested by examining its ability to reduce forced oxidation (Fenton reaction) on ionizable lipid ALC-315. Various formulations with cationic lipid in ethanol and H₂O₂ were tested, with varying concentrations of methionine as set forth in Table 8. The area under the curve (representing the amount of ionizable lipid that was not oxidized) was measured and results are shown in FIG. 10. Methionine performed the best in these experiments from 0.075% w/v to 0.5%.

TABLE 8
Methionine Concentrations Tested

| | Methionine conc |
|---|---|
| Samples | (% w/v) |
| T84-1 | 0.00% |
| T84-b-3 | 0.025% |
| T84-b-4 | 0.050% |
| T84-b-5 | 0.075% |
| T84-2 | 0.15% |
| T84-3 | 0.30% |
| T84-4 | 0.50% |

The effect of cysteine was also examined by comparing a control formulation (T155-1) comprising LNP, 1× TE buffer, and 10% w/v sucrose to a test formulation (T155-4) comprising LNP, 1× TE buffer, 10% w/v sucrose, and 0.25% w/v cysteine. T155-4 resulted in about two times greater stability of LNP encapsulated mRNA in 1 week at 40° C. accelerated conditions.

It was hypothesized that certain excipients, including certain salts, might help prevent or lessen mRNA hydrolysis leading to increased stability. These salts and other excipients set forth in Table 9 were investigated for ability to improve stability of unencapsulated, naked mRNA at high temperature accelerated conditions (60° C.). SARS-CoV-2 mRNA was used at a concentration of 0.1 mg/mL and tested with various excipients. mRNA integrity of each sample was measured using capillary electrophoresis at the start of the experiment and after 18 hours and 24 hours at 60° C.

TABLE 9
Excipients Tested

| Excipients | Category |
|---|---|
| Manganese (II) chloride | Salts |
| Lithium chloride | |
| Magnesium Chloride | |
| Sodium Chloride | |
| Potassium Chloride | |
| Guanidine Thiocyanate | Chaotropic agent |
| Guanidinium chloride | |
| Spermine | Polyamines |
| Spermidine | |
| Ammonium Sulfate | Salts of Kosmotropic Anion |
| Sodium Sulfate | |
| Potassium Sulfate | |
| Polysorbate-20 | Nonionic surfactants |
| Polysorbate-60 | |
| Polysorbate-80 | |
| Sucrose | Sugars |
| D-(+)-Trehalose | |
| Dextrose | |

Various Amino Acids and Derivatives

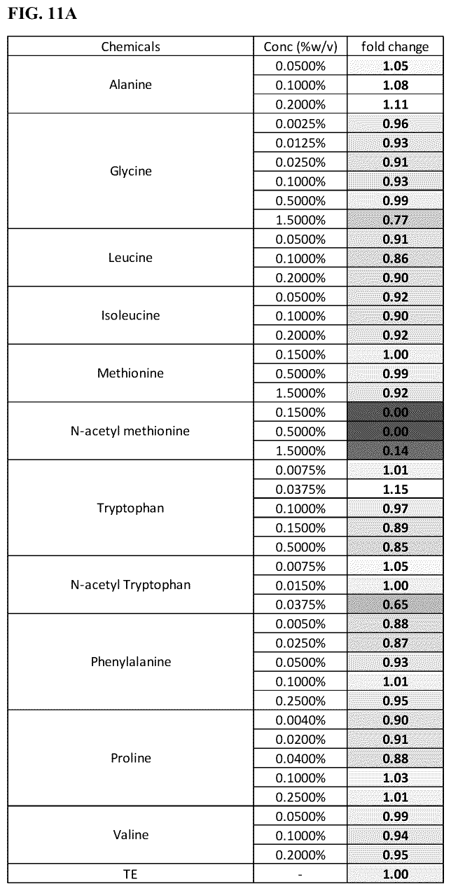

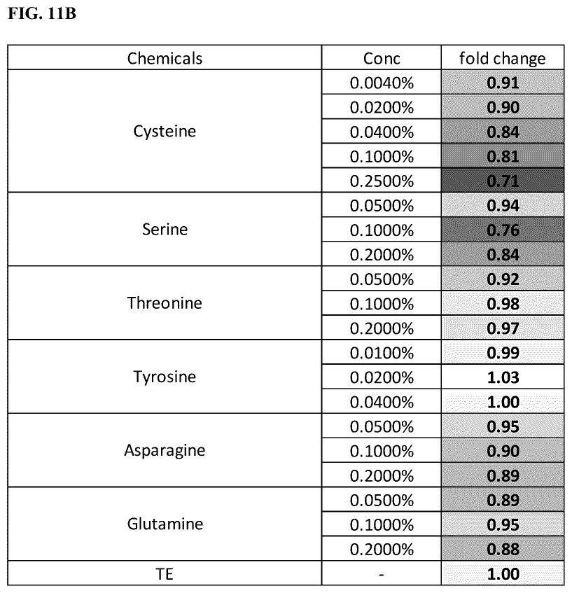

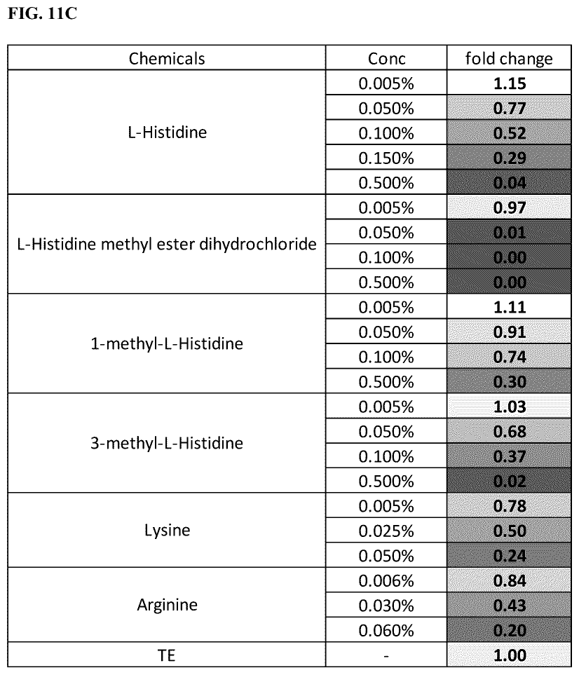

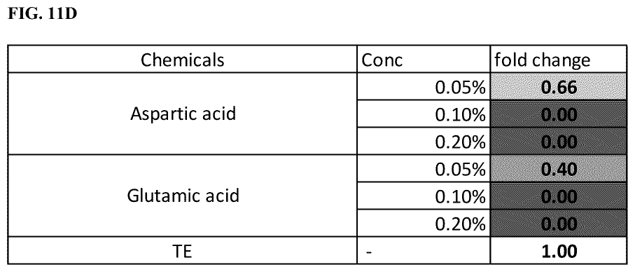

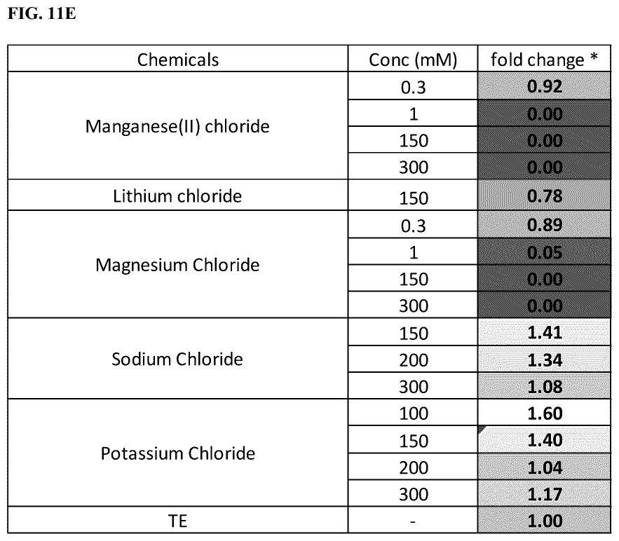

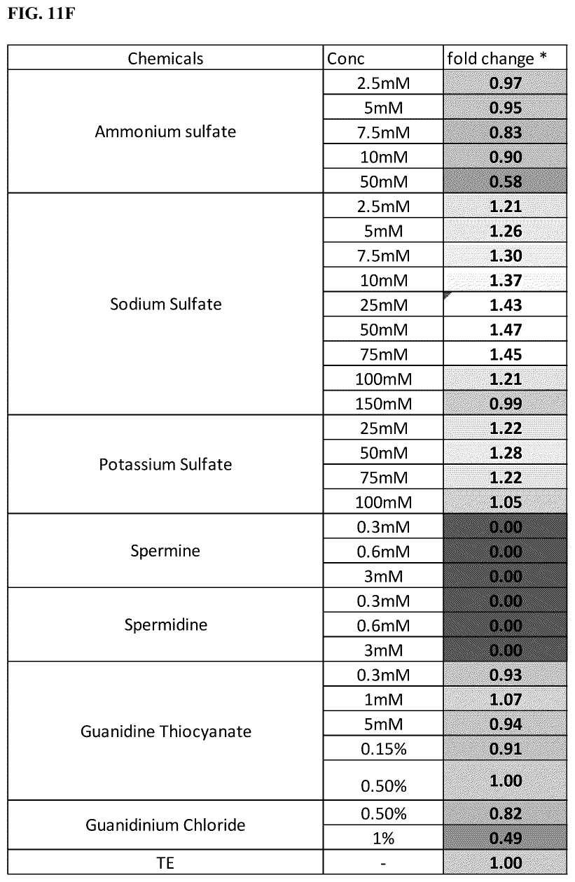

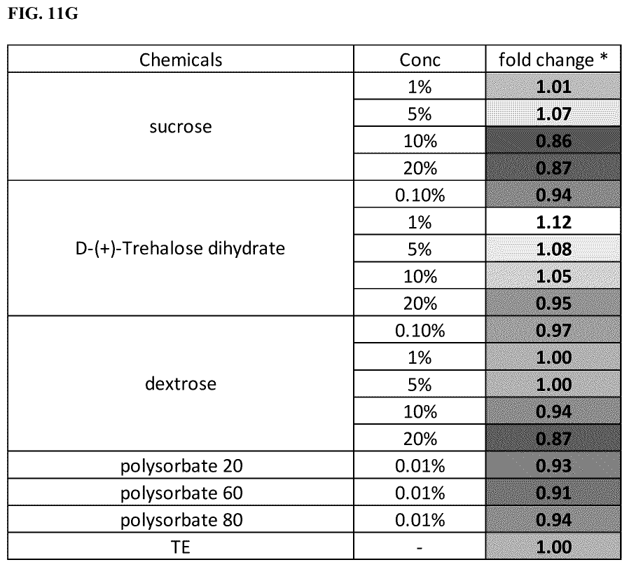

Results are shown in FIGS. 11A (hydrophobic amino acids), 11B (hydrophilic non-charged amino acids), 11C (polar basic amino acids), 11D (polar negatively charged amino acids), 11E and 11F (salts, chaotropic agents, and polyamines) and 11G (sugars and non-ionic surfactants). Improved mRNA integrity was observed with sodium chloride (NaCl) and potassium chloride (KCl) as well as salts of kosmotropic anions, such as sodium sulfate (Na₂SO₄) and potassium sulfate (K₂SO₄) (FIG. 11E). 100 mM to 200 mM NaCl and KCl, and 25 mM to 75 mM sodium sulfate and potassium sulfate, were selected for further study. Hydrophobic amino acids and hydrophilic non-charged amino acids (FIGS. 11A and B) had no positive or negative effect on mRNA integrity in this testing. At lower concentrations polar amino acids (equimolar to moles of mRNA) had no effect but mRNA integrity was reduced at higher concentrations (FIG. 11C and FIG. 11D). Certain salts such as MgCl₂, MnCl₂, LiCl did not help improve mRNA integrity (FIG. 11E). Both spermine and spermidine degraded mRNA significantly (FIG. 11F). Guanidine thiocyanate and guanidinium chloride did not contribute to stability of mRNA while higher concentration of guanidinium chloride had a negative impact (FIG. 11F).

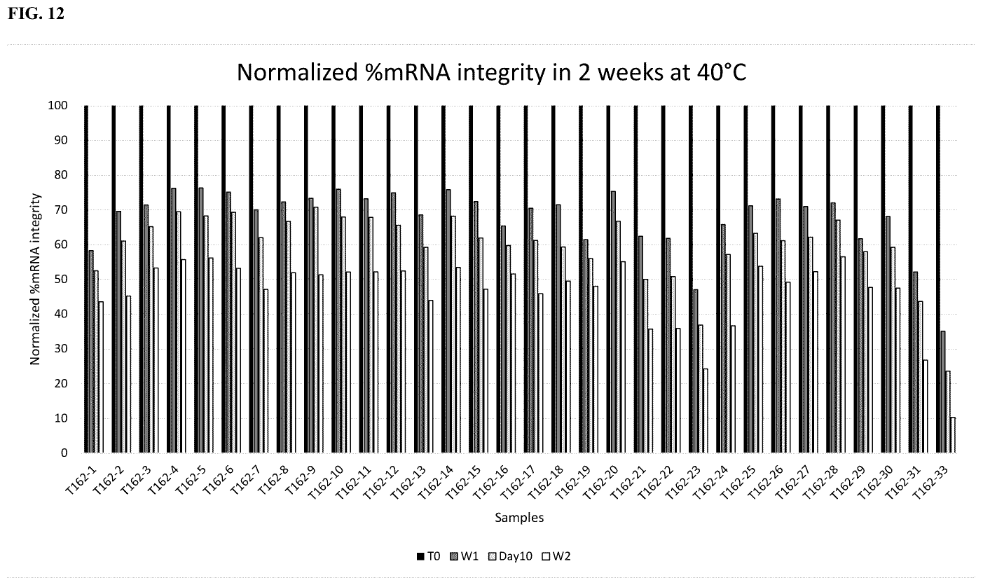

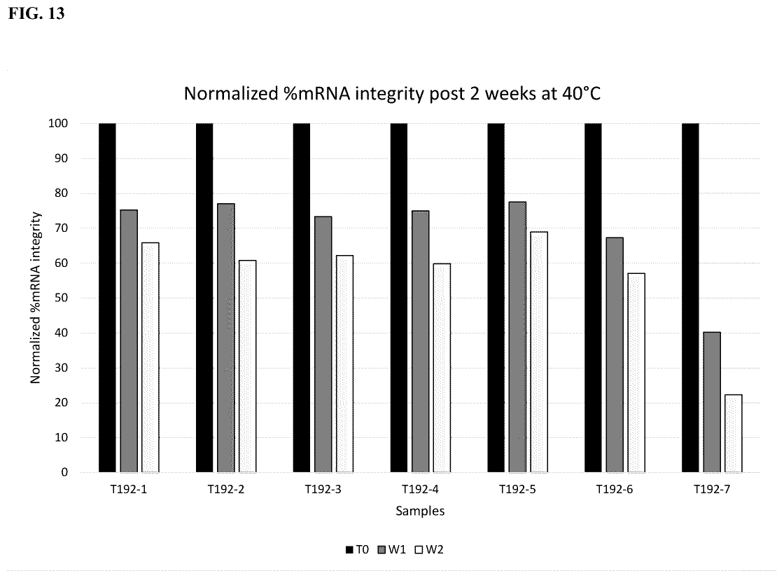

Further experiments were conducted on sodium chloride (NaCl), potassium chloride (KCl), sodium sulfate (Na₂SO₄), and potassium sulfate (K₂SO₄) in various ratios and in combination with various other excipients of the present disclosure with LNP encapsulated SARS-COV-2 mRNA as set forth in Table 10. Normalized % mRNA integrity was measured by HPLC as described in Example 1, after two weeks at 40° C. Results are shown in FIG. 12. All compositions showed improved stability vs. Control (T162-33). Following these initial experiments, additional testing was run on certain samples set forth in Table 11. Normalized % mRNA integrity was measured by HPLC after two weeks at 40° C. Results are shown in FIG. 13. All compositions showed improved stability vs. Control (T192-7).

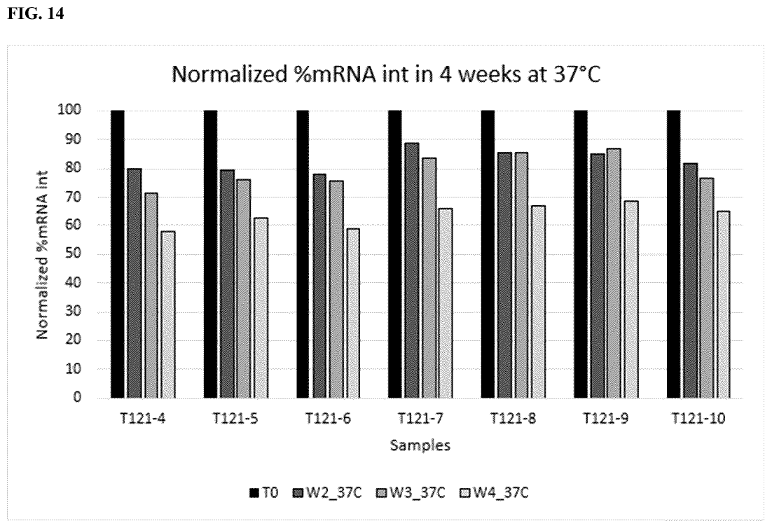

Experiments were also conducted on sodium chloride (NaCl) at various concentrations with lipid-encapsulated luciferase mRNA in buffered solution as set forth in Table 12. mRNA integrity was measured through four weeks at 37° C. and results are set forth in FIG. 14. All concentrations resulted in stable mRNA through week four.

TABLE 10

| | Na₂SO₄ | K₂SO₄ | NaCl | KCl | Cysteine | Methionine | Glycine | Sucr [symbol] |
|---|---|---|---|---|---|---|---|---|
| Samples | (mM) | (mM) | (mM) | (mM) | (% w/v) | (% w/v) | (% w/v) | (% w [symbol] |
| T162-1 | 53 | 0 | 0 | 147 | 0 | 0 | 0 | 10 [symbol] |
| T162-2 | 0 | 74 | 0 | 138 | 0.448 | 0 | 1.5 | 15 [symbol] |
| T162-3 | 73 | 72 | 0 | 197 | 0 | 0 | 1.5 | 10 [symbol] |
| T162-4 | 0 | 33 | 190 | 199 | 0.12 | 0.15 | 0 | 15 [symbol] |
| T162-5 | 71 | 49 | 197 | 145 | 0.15 | 0 | 1.5 | 10 [symbol] |
| T162-6 | 34 | 43 | 136 | 136 | 0.13 | 0 | 0 | 15 [symbol] |
| T162-7 | 66 | 29 | 159 | 183 | 0 | 0 | 1.5 | 15 [symbol] |
| T162-8 | 57 | 50 | 100 | 106 | 0.355 | 0 | 0 | 10 [symbol] |
| T162-9 | 25 | 52 | 120 | 159 | 0.475 | 0 | 0 | 15 [symbol] |
| T162-10 | 75 | 0 | 146 | 200 | 0.463 | 0.15 | 1.5 | 15 [symbol] |
| T162-11 | 54 | 71 | 182 | 192 | 0.5 | 0.15 | 1.5 | 10 [symbol] |
| T162-12 | 75 | 65 | 104 | 110 | 0.117 | 0.15 | 0 | 10 [symbol] |
| T162-13 | 50 | 25 | 0 | 127 | 0.414 | 0.15 | 1.5 | 15 [symbol] |
| T162-14 | 35 | 62 | 171 | 102 | 0.396 | 0.15 | 0 | 15 [symbol] |
| T162-15 | 27 | 71 | 143 | 0 | 0.309 | 0.15 | 1.5 | 10 [symbol] |
| T162-16 | 0 | 47 | 141 | 180 | 0 | 0.15 | 0 | 10 [symbol] |
| T162-17 | 32 | 56 | 0 | 184 | 0.272 | 0 | 1.5 | 10 [symbol] |
| T162-18 | 63 | 59 | 179 | 0 | 0.194 | 0.15 | 1.5 | 10 [symbol] |
| T162-19 | 71 | 0 | 127 | 100 | 0 | 0 | 0 | 15 [symbol] |
| T162-20 | 0 | 67 | 176 | 151 | 0.1 | 0.15 | 0 | 15 [symbol] |
| T162-21 | 0 | 0 | 0 | 0 | 0.49 | 0.15 | 1.5 | 10 [symbol] |
| T162-22 | 67 | 33 | 0 | 0 | 0.283 | 0 | 1.5 | 15 [symbol] |
| T162-23 | 0 | 39 | 0 | 0 | 0 | 0.15 | 0 | 15 [symbol] |
| T162-24 | 0 | 0 | 157 | 118 | 0.292 | 0.15 | 1.5 | 15 [symbol] |
| T162-25 | 58 | 75 | 147 | 124 | 0 | 0.15 | 1.5 | 15 [symbol] |
| T162-26 | 30 | 34 | 200 | 0 | 0.457 | 0 | 0 | 15 [symbol] |
| T162-27 | 48 | 0 | 192 | 0 | 0.1 | 0 | 0 | 10 [symbol] |
| T162-28 | 0 | 0 | 106 | 193 | 0.21 | 0.15 | 0 | 10 [symbol] |
| T162-29 | 37 | 0 | 100 | 100 | 0 | 0.15 | 1.5 | 10 [symbol] |
| T162-30 | 0 | 37 | 100 | 100 | 0.5 | 0 | 1.5 | 10 [symbol] |
| T162-31 | 0 | 0 | 0 | 0 | 0 | 0.15 | 1.5 | 0 [symbol] |
| T162-33 | 0 | 0 | 0 | 0 | 0 | 0 | 0 | 10 [symbol] |

[symbol]

indicates data missing or illegible when filed

TABLE 11

| Samples | Contents |
|---|---|
| T192-1 | LNP, 33 mM K₂SO₄, 190 mM NaCl, 199 mM KCl, 0.12% w/v |
| | cysteine, 0.15% w/v methionine, 10% w/v sucrose, 0.01 [symbol] |
| T192-2 | LNP, 34 mM Na₂SO₄, 43 mM K₂SO₄, 136 mM NaCl, 136 mM |
| | KCl, 0.13% w/v cysteine, 10% w/v sucrose, 0.01% w/v [symbol] |
| T192-3 | LNP, 47 mM K₂SO₄, 141 mM NaCl, 180 mM KCl, 0.15% w/v |
| | methionine, 10% w/v sucrose, 0.01% w/v PS-20, TE |
| T192-4 | LNP, 48 mM Na₂SO₄, 192 mM NaCl, 0.1% w/v cysteine, |
| | 10% w/v sucrose, 0.01% w/v PS-20, TE |
| T192-5 | LNP, 75 mM Na₂SO₄, 75 mM K₂SO₄, 200 mM NaCl, 200 mM |
| | KCl, 10% w/v sucrose, 0.01% w/v PS-20, TE |
| T192-6 | LNP, 75 mM Na₂SO₄, 200 mM NaCl, 125 mM KCl, 10% w/v |
| | sucrose, 0.01% w/v PS-20, TE |
| T192-7 | LNP, Sucrose, PBS |

[symbol]

indicates data missing or illegible when filed

TABLE 12

| Samples | Buffer |
|---|---|
| T121-4 | 1X TE Buffer with 10 mM |
| | Sodium Chloride |
| T121-5 | 1X TE Buffer with 50 mM |
| | Sodium Chloride |
| T121-6 | 1X TE Buffer with 100 mM |
| | Sodium Chloride |
| T121-7 | 1X TE Buffer with 150 mM |
| | Sodium Chloride |
| T121-8 | 1X TE Buffer with 300 mM |
| | Sodium Chloride |
| T121-9 | 1X TE Buffer with 600 mM |
| | Sodium Chloride |
| T121-10 | 1X TE Buffer with 1000 mM |
| | Sodium Chloride |

1. A lipid nanoparticle (LNP)-encapsulated RNA composition, comprising an LNP-encapsulated RNA and a combination of excipients providing stability for at least three months with refrigeration in the range of 2° C.-8° C.

2. The composition of claim 1, wherein the composition is stable for at least three months at 2° C.

3. The composition of claim 1, wherein the composition is stable for at least six months at 2° C.

4. The composition of claim 1, wherein the composition is stable for at least three months at 4° C.

5. The composition of claim 1, wherein the composition is stable for at least six months at 4° C.

6. The composition of claim 1, wherein the composition is stable for at least three months at 8° C.

7. The composition of claim 1, wherein the composition is stable for at least six months with refrigeration in the range of 2° C.-8° C.

8. The composition of claim 1, wherein at least 60% of the RNA remains intact after 6 months at 2-8° C., as compared to the same composition at time zero, optionally wherein at least 70% of the RNA remains intact after 6 months at 2-8° C., as compared to the same composition at time zero, optionally wherein at least 80% of the RNA remains intact after 6 months at 2-8° C., as compared to the same composition at time zero, optionally wherein at least 90% of the RNA remains intact after 6 months at 2-8° C., as compared to the same composition at time zero.

9. The composition of any one of claims 1 to 8, wherein the excipients comprise an antioxidant, a non-ionic surfactant, a stabilizing agent, and a pH buffer.

10. The composition of claim 9, wherein the antioxidant is selected from one or more of methionine, cysteine, and ascorbic acid.

11. The composition of claim 10, wherein the antioxidant is methionine or cysteine.

12. The composition of claim 11, wherein the antioxidant is methionine, and the concentration of methionine is optionally from about 0.05% to about 1.50% w/v, or from about 0.05% to about 0.5% w/v, or from or about 0.05% to about 0.25% w/v, or from about

0. 05% to about 0.15% w/v.

13. The composition of any one of claims 9 to 12, wherein the non-ionic surfactant is a polysorbate, optionally polysorbate-20.

14. The composition of claim 13, wherein the composition comprises a polysorbate at a concentration of about 0.001% to about 0.1% w/v, or from about 0.005% to about 0.05% w/v, or about 0.01% w/v, and which is optionally polysorbate-20.

15. The composition of any one of claims 9 to 12, wherein the non-ionic surfactant is a poloxamer, and optionally Poloxamer 188.

16. The composition of claim 15, wherein the composition comprises the poloxamer at a concentration of about 0.001% to about 0.1% w/v, or from about 0.005% to about 0.05% w/v, or about 0.01% w/v.

17. The composition of any one of claims 9 to 16, wherein the stabilizing agent is selected from one or more of glycine, sorbitol, and gelatin.

18. The composition of claim 17, wherein the stabilizing agent is glycine.

19. The composition of claim 18, wherein the concentration of glycine is from about 0.25% to about 15% w/v, or from about 0.5% to about 15% w/v, or from about 0.25% to about 10% w/v, or from about 0.25% to about 5% w/v, or from about 0.5% to about 2.5% w/v, or about 1.5% w/v.

20. The composition of claim 19, comprising or consisting essentially of the following excipients in buffered solution:
methionine from about 0.05% to about 1.50% w/v;
polysorbate-20 from about 0.001% to about 0.1% w/v; and
glycine from about 0.25% to about 15% w/v.

21. The composition of claim 20, comprising or consisting essentially of the following excipients in buffered solution:
methionine from about 0.05 to about 0.25% w/v;
polysorbate-20 from about 0.005% to about 0.05% w/v; and
glycine from about 0.5% to about 2.5% w/v.

22. The composition of claim 21, comprising or consisting essentially of the following excipients in buffered solution: methionine at about 0.075 to about 0.15% w/v, polysorbate-20 at about 0.01% w/v, and glycine at about 1.5% w/v.

23. The composition of any one of claims 1 to 22, wherein the composition is pH buffered in the range of about 6.0 to about 8.0.

24. The composition of claim 23, wherein the composition is buffered at about pH 7.4.

25. The composition of claim 23 or 24, wherein the pH buffer is a phosphate buffer.

26. The composition of claim 23 or 24, wherein the pH buffer is selected from a Tris-EDTA buffer, a histidine buffer, and a sodium citrate buffer.

27. The composition of claim 26, wherein the pH buffer is Tris-EDTA buffer, optionally wherein the Tris-EDTA buffer is at a concentration of 1× to 10×, optionally wherein the Tris-EDTA buffer is at a concentration of 1× to 5×, optionally wherein the Tris-EDTA buffer is at a concentration of 1× to 3×, optionally wherein the Tris-EDTA buffer is a concentration of 1×.

28. The composition of any one of claims 1 to 27, wherein the composition comprises or further comprises a metal ion chelator.

29. The composition of claim 28, wherein the metal ion chelator is EDTA or salt thereof.

30. The composition of claim 29, wherein the metal ion chelator is disodium EDTA.

31. The composition of claim 29 or 30, wherein the concentration or EDTA or disodium EDTA is from about 0.01 mM to about 1 mM, or from about 0.05 mM to about 0.5 mM, or about 0.1 mM.

32. The composition of any one of claims 1 to 31, wherein the composition further comprises an excipient that reduces exposure of the RNA to water.

33. The composition of claim 32, wherein the excipient that reduces exposure of the mRNA to water is sucrose.

34. The composition of any one of claims 1 to 33, wherein the composition further comprises an excipient that reduces degradation of the RNA by free-radical oxidation.

35. The composition of claim 34, wherein the excipient that reduces degradation of the RNA by free-radical oxidation is one or more of ethanol and histidine.

36. The composition of claim 35, wherein ethanol is included as an excipient at 200 mM or less, or about 150 mM or less, or about 100 mM of less, or about 50 mM or less.

37. The composition of any one of claims 34 to 36, wherein the composition comprises histidine at a concentration of from about 0.01% w/v to about 1% w/v, or from about 0.05% w/v to about 0.5% w/v, or about 0.1% w/v.

38. The composition of any of claims 1 to 8, wherein the excipients comprise one or more salts, and a pH buffer.

39. The composition of claim 38, further comprising one or more additional excipients selected from a non-ionic surfactant, antioxidants, stabilizing agents, and cryoprotectants.

40. The composition of claim 38 or 39, wherein the one or more salts are selected from the group of sodium chloride, potassium chloride, sodium sulfate, and potassium sulfate.

41. The composition of claim 40, wherein the one or more salts comprise at least one chloride salt and at least one sulfate salt; and/or wherein the one or more salts comprise at least one potassium salt and at least one sodium salt.

42. The composition of claim 40, wherein the one or more salts comprise or consist essentially of:
(a) sodium sulfate, potassium chloride, and sodium chloride;
(b) sodium chloride, potassium chloride, sodium sulfate, and potassium sulfate;
(c) potassium sulfate, sodium chloride, and potassium chloride;
(d) sodium chloride, sodium sulfate, and potassium sulfate;
(e) sodium chloride and potassium sulfate;
(f) sodium chloride and potassium chloride;
(g) potassium chloride, potassium sulfate, and sodium sulfate;
(h) potassium chloride and potassium sulfate;
(i) sodium sulfate and sodium chloride;
(j) potassium chloride and sodium sulfate;
(k) potassium sulfate and sodium sulfate;
(l) sodium chloride;
(m) potassium chloride;
(n) sodium sulfate; or
(o) potassium sulfate.

43. The composition of claim 40, wherein the one or more salts comprise or consist essentially of potassium sulfate, sodium chloride, and potassium chloride.

44. The composition of claim 40, wherein the one or more salts comprise or consist essentially of sodium sulfate and sodium chloride.

45. The composition of any one of claims 40 to 44, wherein the concentration of one or more sulfate salts is from about 2.5 mM to about 150 mM, or from about 10 mM to about 75 mM, or from about 25 mM to about 75 mM, or from about 33 mM to about 75 mM.

46. The composition of any of claims 40 to 44, wherein the concentration of one or more chloride salts is from about 10 mM to about 1,000 mM, or from about 50 mM to about 600 mM, or from about 100 mM to about 500 mM, or is from about 100 mM to about 200 mM, or is from about 136 mM to about 200 mM.

47. The composition of claim 45 or 46, wherein the concentration of potassium sulfate is about 43 mM, the concentration of sodium sulfate is about 34 mM, the concentration of sodium chloride is about 136 mM, and the concentration of potassium chloride is about 136 mM.

48. The composition of claim 45 or 46, wherein the concentration of potassium sulfate is about 75 mM, the concentration of sodium sulfate is about 75 mM, the concentration of sodium chloride is about 200 mM, and the concentration of potassium chloride is about 200 mM.

49. The composition of claim 45 or 46, wherein the concentration of potassium sulfate is about 33 mM, the concentration of potassium chloride is about 199 mM, and the concentration of sodium chloride is about 190 mM.

50. The composition of claim 45 or 46, wherein the concentration of potassium sulfate is about 47 mM, the concentration of potassium chloride is about 180 mM, and the concentration of sodium chloride is about 141 mM.

51. The composition of claim 45 or 46, wherein the concentration of sodium sulfate is about 48 mM and the concentration of sodium chloride is about 192 mM.

52. The composition of claim 45 or 46, wherein the concentration of the sodium sulfate is about 75 mM, the concentration of potassium chloride is about 125 mM, and the concentration of sodium chloride is about 200 mM.

53. The composition of any of claims 39 to 52, wherein the composition comprises a nonionic surfactant, optionally wherein the nonionic surfactant is selected from polysorbate-20, polysorbate-60, and polysorbate-80.

54. The composition of claim 53, wherein the nonionic surfactant is in a concentration of about 0.005% to about 0.04%, optionally wherein the concentration is about 0.01% w/v, and the nonionic surfactant is optionally polysorbate-20.

55. The composition of any of claims 38 to 54, wherein the pH buffer is selected from Tris-EDTA (TE), PBS, histidine, Tris acetate, and sodium citrate.

56. The composition of claim 55, wherein the pH buffer is Tris-EDTA (TE) buffer, optionally wherein the Tris-EDTA (TE) buffer is at a concentration of 1× to 10×, optionally wherein the Tris-EDTA (TE) buffer is at a concentration of 1× to 5×, optionally wherein the Tris-EDTA (TE) buffer is at a concentration of 1× to 3×, optionally wherein the Tris-EDTA (TE) buffer is a concentration of 1×.

57. The composition of any of claims 39 to 56, wherein the composition comprises one or more antioxidants, optionally wherein the one or more antioxidants is selected from methionine and cysteine.

58. The composition of claim 57, wherein the one or more antioxidants comprises cysteine, optionally wherein the cysteine is in a concentration of about 0.05% to about 0.5%, or from about 0.075% to about 0.5%, or from about 0.10% to about 0.5%, or from about 0.10% to about 0.20%, or about 0.10% to about 0.15% w/v.

59. The composition of claim 57 or 58, wherein the one or more antioxidants comprises methionine, optionally wherein the methionine is in a concentration of from about 0.025% to about 3.0% w/v, or from about 0.025% to about 0.5% w/v, or from about 0.075% to about 0.5%, or from about 0.05 to about 0.25% w/v, or about 0.15% w/v.

60. The composition of any one of claims 39 to 59, wherein the composition comprises a cryoprotectant, optionally wherein the cryoprotectant is sucrose, optionally wherein the sucrose is in a concentration of about 5% to about 20%, optionally wherein the sucrose is in a concentration from about 10% to about 15% w/v, optionally wherein the sucrose is in a concentration of about 10%.

61. The composition of any one of claims 39 to 60, wherein the composition comprises a stabilizing agent, optionally wherein the stabilizing agent is selected from one or more of glycine, sorbitol, and gelatin.

62. The composition of any claim 62 wherein the stabilizing agent is glycine.

63. The composition of claim 61 or 62 wherein the stabilizing agent is at a concentration of about 0.05% to about 15% w/v, optionally wherein the stabilizing agent is at a concentration of about 0.5% to about 10% w/v, optionally wherein the stabilizing agent is at a concentration of about 1% to about 2%, optionally wherein the stabilizing agent is at a concentration of about 1.5%.

64. The composition of any one of claims 1 to 63, wherein the RNA is messenger RNA (mRNA).

65. The composition of claim 64, wherein the RNA is an mRNA encoding a component of an infectious agent.

66. The composition of claim 65, wherein the infectious agent is a virus.

67. The composition of claim 66, wherein the virus is a coronavirus, such as a betacoronavirus.

68. The composition of claim 67, wherein the betacoronavirus is SARS-COV.

69. The composition of any one of claims 66 to 68, wherein the mRNA encodes one or more viral structural proteins or one or more polypeptides derived from virus proteins.

70. The composition of claim 69, wherein the mRNA encodes a coronavirus Spike protein(S).

71. The composition of claim 69 or claim 70, wherein the mRNA encodes M (membrane) glycoprotein, E (envelope) protein, and/or N (nucleocapsid) protein.

72. The composition of claim 66, wherein the mRNA encodes one or more influenza proteins, such as neuraminidase (NA), hemagglutinin (HA), matrix protein 2 (M2), and/or nucleoprotein (NP).

73. The composition of claim 64, wherein the mRNA encodes a therapeutic protein.

74. The composition of claim 66, wherein the RNA encodes a varicella zoster protein, optionally wherein the varicella zoster protein is selected from glycoprotein E and glycoprotein I.

75. The composition of any one of claims 64 to 74, wherein the composition is injectable.

76. A method for expressing an mRNA in cells of a patient, comprising, administering the composition of any one of claims 1 to 75 in said patient.

77. A method for expressing an mRNA in cells of a mammal, comprising, administering the composition of any one of claims 1 to 75 in said mammal, optionally wherein the mammal is selected from the group consisting of a pig and a cow.

78. A method for expressing an mRNA in cells of a bird, comprising. administering the composition of any one of claims 1 to 75 in said bird, optionally wherein the bird is a chicken.

79. A method for increasing the stability of an RNA by formulating the RNA in a composition of any of claims 1 to 75.
